(19) United States
(12) Patent Application Publication
(10) Pub. No.: US 2026/0247068 A1
(43) Pub. Date: Aug. 20, 2026

(54) DANGEROUS AREA NOTIFICATION APPARATUS

(71) Applicant: NTT, Inc., Tokyo (JP)

(72) Inventors: Tatsuya KAKO, Musashino-shi, Tokyo (JP); Hironobu CHIBA, Musashino-shi, Tokyo (JP); Ryosuke SUGIURA, Musashino-shi, Tokyo (JP)

(73) Assignee: NTT, Inc., Tokyo (JP)

(21) Appl. No.: 19/159,333

(22) PCT Filed: Mar. 1, 2023
(86) PCT No.: PCT/JP2023/007631

§ 371 Date: Aug. 25, 2025

(51) Int. Cl. H04R 1/34; A61F 9/08; A61H 3/06; B61L 29/24; E01C 9/00; H04R 1/28; H04R 1/40; H04R 27/00

(52) U.S. Cl. CPC H04R 1/345; A61F 9/08; A61H 3/066; B61L 29/243; H04R 1/2811; H04R 27/00; E01C 9/00; H04R 1/403

(57) ABSTRACT

There is a problem that it is difficult for a visually impaired person to immediately judge whether or not he/she is in a railroad crossing or crosswalk based on only tactile paving. In order to solve this problem, a dangerous area notification apparatus according to the disclosed technology is a speaker that emits a sound toward a sidewalk or its periphery in a railroad crossing, and emits a sound different from an alarm sound of a railroad crossing gate.

### TECHNICAL FIELD

The disclosed technology relates to an apparatus that delivers sound only to a desired position and range in a railroad crossing.

### BACKGROUND ART

Tactile paving (NPL 1) is installed on the railroad crossing or a crosswalk so that visually impaired persons can walk safely.

Our country's tactile paving installation guideline defines that tactile paving should be placed around the railroad crossing or the crosswalk. Specifically, tactile paving in which the person can perceive presence and direction of the railroad crossing and the crosswalk are placed in front of the railroad crossing or the crosswalk. In addition, tactile paving is placed so that the person can perceive that he/she is on a railroad track (in the railroad crossing) or on a road (in the crosswalk) at the railroad crossing or the crosswalk.

### PRIOR ART LITERATURE

### Non-Patent Literature

[NPL 1]B. Bentzen et al., “Tactile Walking Surface Indicators in the United States and Internationally”, 2021.

### SUMMARY OF THE INVENTION

### Problems to be Solved by the Invention

However, it is sometimes difficult to immediately judge whether or not the location is in the railroad crossing or the crosswalk based on only tactile paving.

An object of the present disclosed technology is to enable the visually impaired person to more easily know that the person is in an area that is dangerous to remain in such as a railroad crossing or crosswalk, and that he/she has left the dangerous area.

### Means to Solve the Problems

In order to solve the above-described problem, a dangerous area notification apparatus according to the disclosed technology is a speaker that emits a sound toward a sidewalk in a railroad crossing or its periphery, and emits a sound different from an alarm sound of a railroad crossing gate.

### Effects of the Invention

According to the disclosed technology, a visually impaired person can more easily know whether or not the person is in the area that is dangerous to remain in such as a railroad crossing or crosswalk.

### BRIEF DESCRIPTION OF THE DRAWINGS

FIG. 1 is a transparent front view illustrating a configuration of an acoustic signal output apparatus as a first embodiment of a localized sound speaker.

FIG. 2 is a transparent plane view illustrating the configuration of the acoustic signal output apparatus as the first embodiment of the localized sound speaker.

FIG. 3 is a 1-1 cross-sectional view of FIG. 1.

FIG. 4 is a 2-2 cross-sectional view of FIG. 2.

FIG. 5 is a conceptual diagram for illustrating an arrangement of sound holes.

FIG. 6 is a conceptual diagram for explaining relation between a paraboloid of revolution and a focal point.

FIG. 7A is a conceptual diagram for explaining a traveling direction of an acoustic signal when a driver unit is placed at the focal point of the paraboloid of revolution. FIG. 7B is a conceptual diagram for explaining the traveling direction of the acoustic signal when the driver unit is not placed at the focal point of the paraboloid of revolution.

FIG. 8A is a conceptual diagram for illustrating a configuration in which a horn is attached to the driver unit. FIG. 8B is a conceptual diagram for illustrating a configuration of the acoustic signal output apparatus as the first embodiment of the localized sound speaker.

FIG. 9A is a block diagram for illustrating a functional configuration for providing a signal to the driver unit. FIG. 9B is a diagram illustrating a sound pressure level at an observation point.

FIG. 10A and FIG. 10B are graphs for illustrating a directional characteristic of the acoustic signal output apparatus.

FIG. 11A and FIG. 11B are graphs for illustrating the directional characteristic of the acoustic signal output apparatus.

FIG. 12 is a graph for illustrating the directional characteristic of the acoustic signal output apparatus.

FIG. 13A and FIG. 13B are graphs for illustrating a frequency characteristic of the acoustic signal output apparatus.

FIG. 14A and FIG. 14B are graphs for illustrating a frequency characteristic of the acoustic signal output apparatus.

FIG. 15 is a graph for illustrating the frequency characteristic of the acoustic signal output apparatus.

FIG. 16 is a cross-sectional view for illustrating a modification example of the arrangement of sound holes.

FIG. 17 is a cross-sectional view for illustrating a modification example of the arrangement of sound holes.

FIG. 18 is a transparent front view illustrating a configuration of an acoustic signal output apparatus as a modification example of the first embodiment of the localized sound speaker.

FIG. 19 is a transparent plane view illustrating the configuration of the acoustic signal output apparatus as the modification example of the first embodiment of the localized sound speaker.

FIG. 20A is a transparent plane view illustrating a configuration of a housing of the modification example of the first embodiment of the localized sound speaker. FIG. 20B is a transparent front view illustrating the configuration of the housing of the modification example of the first embodiment of the localized sound speaker. FIG. 20C is a bottom view illustrating the configuration of the housing of the modification example of the first embodiment of the localized sound speaker.

FIG. 21 is a 19-19 cross-sectional view of FIG. 19.

FIG. 22A and FIG. 22B are cross-sectional views for illustrating the configuration of the acoustic signal output apparatus as the modification example of the first embodiment of the localized sound speaker.

FIG. 23 is a transparent front view illustrating a configuration of an acoustic signal output apparatus as a second embodiment of the localized sound speaker.

FIG. 24 is a transparent plane view illustrating the configuration of the acoustic signal output apparatus as the second embodiment of the localized sound speaker.

FIG. 25 is a transparent front view illustrating a configuration of an acoustic signal output apparatus as a modification example of the second embodiment of the localized sound speaker.

FIG. 26 is a transparent front view illustrating the configuration of the acoustic signal output apparatus as the modification example of the second embodiment of the localized sound speaker.

FIG. 27A is a graph for illustrating the frequency characteristic of the acoustic signal observed at a notch part side. FIG. 27B is a graph for illustrating the frequency characteristic of the acoustic signal observed at a side where the notch part is not provided.

FIG. 28A is a graph for illustrating the frequency characteristic of the acoustic signal observed at the notch part side and the side where the notch part is not provided. FIG. 28B is a graph for illustrating a difference in the frequency characteristic of the acoustic signals due to a difference in the notch part.

FIG. 29 is a transparent front view illustrating a configuration of an acoustic signal output apparatus as a third embodiment of the localized sound speaker.

FIG. 30A is a block diagram for illustrating a functional configuration for providing a signal to the driver unit. FIG. 30B is a diagram illustrating the sound pressure level at the observation point.

FIG. 31 is a diagram for explaining an example of a railroad crossing in which a dangerous area notification apparatus is installed.

FIG. 32 is a diagram for explaining the dangerous area notification apparatus according to the first embodiment.

FIG. 33 is a perspective diagram of the railroad crossing 31 in which the dangerous area notification apparatus is installed.

FIG. 34 is a diagram for explaining a notification area by a notification speaker of the first embodiment.

FIG. 35 is a diagram for explaining a case where the first embodiment is applied to a double track.

FIG. 36 is a diagram for explaining a dangerous area notification apparatus according to a second embodiment.

FIG. 37 is a diagram for explaining a dangerous area notification apparatus according to a third embodiment.

FIG. 38 is a diagram for explaining a dangerous area notification apparatus according to a fourth embodiment.

FIG. 39 is a functional block diagram of a dangerous area notification apparatus according to a modification example from the first embodiment to the fourth embodiment.

FIG. 40 is a diagram for explaining a dangerous area notification apparatus according to a fifth embodiment.

### DETAILED DESCRIPTION OF THE EMBODIMENTS

Hereinafter, embodiments of a disclosed technology will be described in detail. Note that constituent units that have the same functions are denoted by the same reference signs, and repeated description thereof will be omitted.

In the following, a dangerous area notification apparatus according to the disclosed technology will be described first, and an acoustic apparatus optimum for the dangerous area notification apparatus will be described.

### First Embodiment

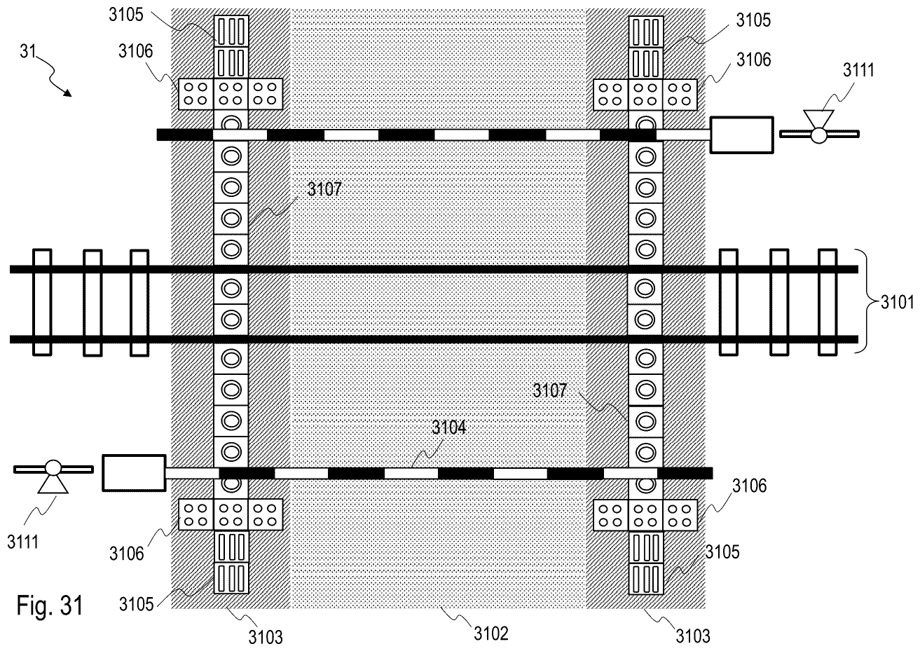

FIG. 31 is a diagram showing an example of a railroad crossing 31 in which a dangerous area notification apparatus according to the disclosed technology is installed.
3101 denotes a railroad track and FIG. 31 shows a case of single track.
3102 denotes a road crossing the railroad track and 3103 denotes a sidewalk crossing the railroad track.

Tactile paving 3105, 3106, and 3107 having different tactile shapes are installed on the sidewalk 3103.

The tactile paving 3105 is provided with tactile shape indicating that there is a railroad crossing ahead, for example.

The tactile paving 3106 is provided with tactile shape indicating that this area ahead is in the railroad crossing, for example.

The tactile paving 3107 is provided with tactile shape indicating that the location is in the railroad crossing, for example.

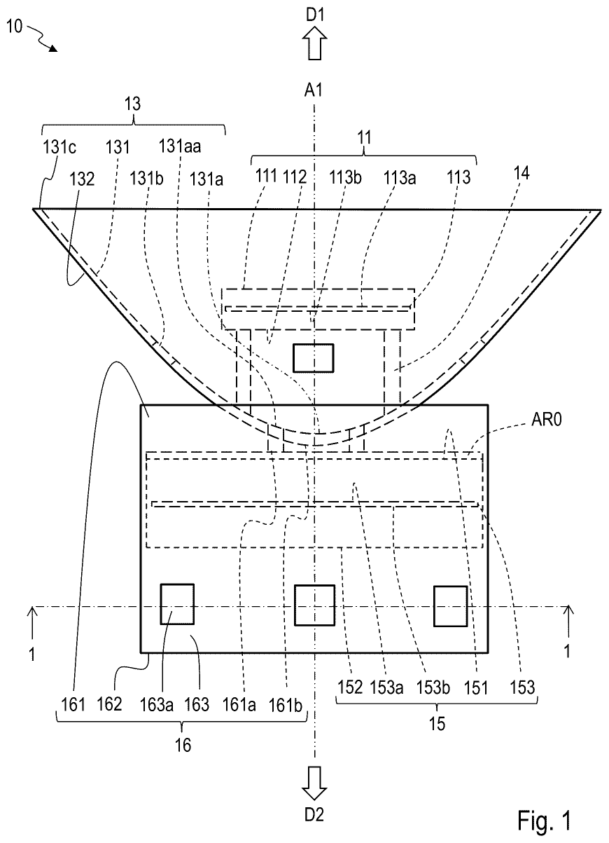

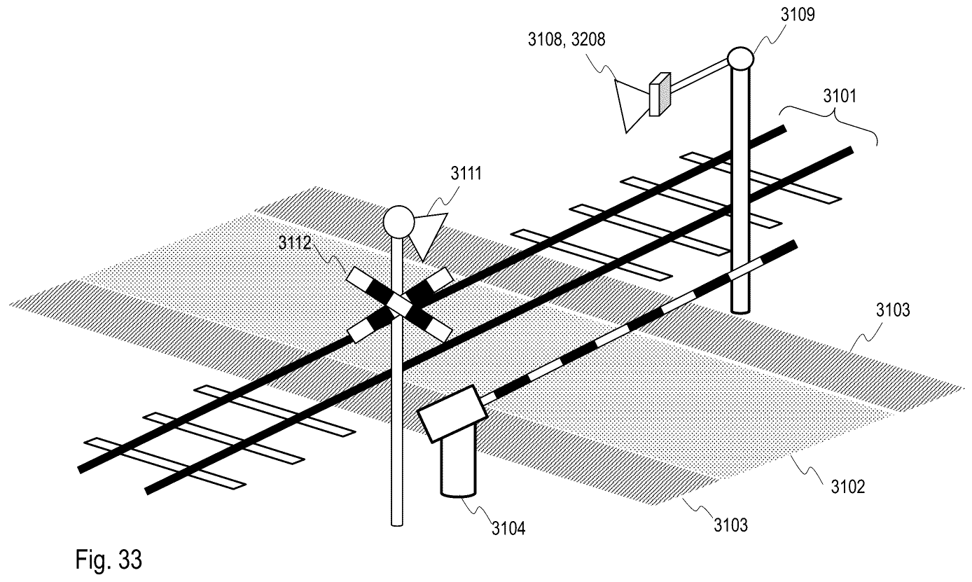

Note that the shape of the tactile paving shown in the figure is one example, and it is sufficient to have a shape different for each function of the tactile paving.
3104 denotes a railroad crossing gate. FIG. 1 shows a state in which the railroad crossing gate is closed.
3111 denotes an alarm speaker which rings when a train approaches and passes in conjunction with the railroad crossing gate 3104, and is mounted on a railroad crossing alarm sign 3112 as shown in FIG. 33, for example. A sound emitted from the alarm speaker 3111 is called as “an alarm sound”.

Note that the alarm speaker 3111 is different from a localized sound speaker 3108 as described later and emits the alarm sound which can be clearly heard in a wide range of the inside and outside of the railroad crossing.

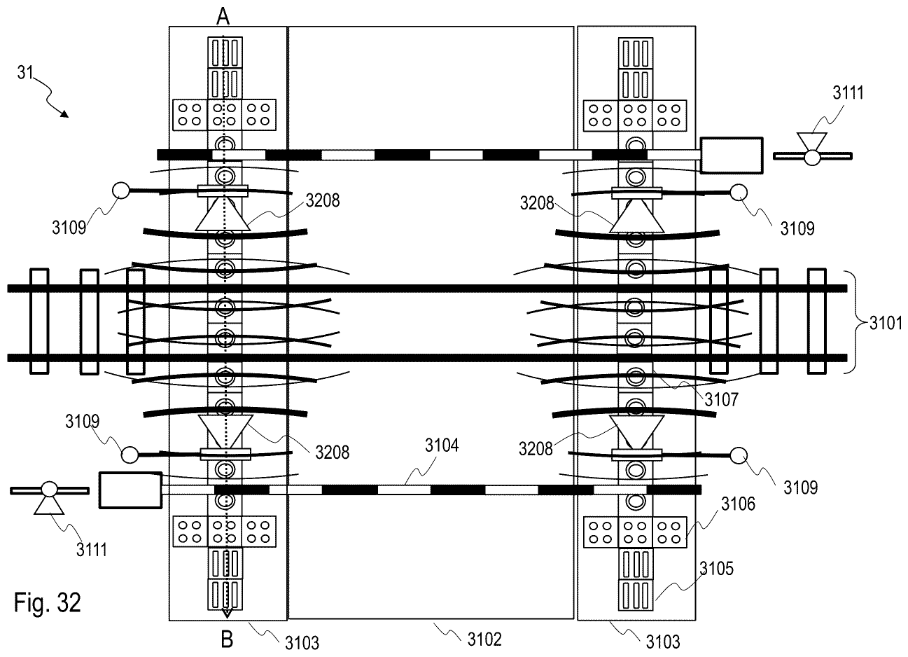

FIG. 32 shows an example in which the dangerous area notification apparatus according to the first embodiment is applied to the railroad crossing 31.
3208 denotes a speaker (notification speaker) for playing a notification sound to inform pedestrians of being in a dangerous area, and 3109 is a support column for supporting the notification speaker 3208 in the air. In the first embodiment, four notification speakers 3208 are used for playing the same notification sound. As illustrated in FIG. 33, the notification speaker 3208 is placed at a height which does not interfere with the passage of an automatic person or a pedestrian.

As the notification speaker 3208, a speaker having at least some degree of directivity is used. Specifically, a cone type speaker or a horn type speaker having higher directivity may be used.

Note that “directivity” means a property in which transmission intensity of the sound outputted into the air differs depending on the direction, and the speaker having directivity is a speaker that limits the direction and width of the sound and can output a strong sound in the specific direction. In addition, the opening direction of the cone or horn is called the front of the speaker, and the inverse side is called the rear of the speaker.

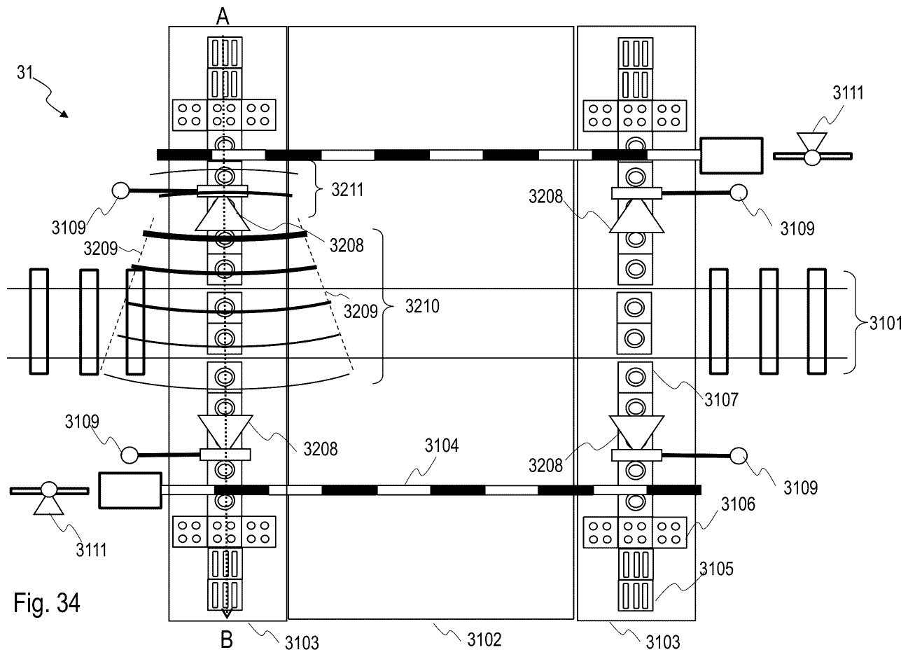

Referring to FIG. 34, a sound area (notification sound area) emitted by the notification speaker 3208 will be described. In FIG. 34, only one of the four notification sound areas is extracted and illustrated for explanation. For visibility of the notification sound area, the railroad track 3101 is also illustrated thin.
3209, 3210 and 3211 show the approximate spread of the notification sound area. A pair of dotted lines 3209 indicates the approximate directivity to be satisfied by the notification speaker 3208, and the direction in which the notification sound reaches is so adjusted that the notification sound can be heard by covering the sidewalk 3103.

Thickness of a gentle arc line group indicated by 3210 indicates intensity of the notification sound. Although the notification sound is naturally attenuated as moving away from the speaker, one sidewalk is covered with two notification speakers 3208 as shown in FIG. 32, and sound volume of each speaker 3208 is adjusted so that the notification sound can be clearly heard by adding the sounds of the two speakers at the middle of the railroad crossing.

Note that the notification sound also comes around behind the speaker 3208, but coming around sound 3211 is more quickly attenuated than the front sound of the notification speaker 3208.

Returning to FIG. 32, operations of the dangerous area notification apparatus will be described using FIG. 32.

### <When No Train is Passing>

The notification sound is played by the notification speaker 3208. The notification sound may be a voice announcement or a non-voice such as a buzzer sound or a chime sound.

When the pedestrian moves from a point A to a point B, the pedestrian experiences the following. Note that it is assumed that the railroad crossing gate is not closed. In addition, it is also assumed that the same notification sound is played by the four notification speakers 3208.

When the pedestrian approaches the railroad crossing, the sound (notification sound) of the point A side notification speaker 3208 is heard. The notification sound becomes larger as approaching the speaker 3208, and when the pedestrian reaches the front of the speaker 3208, the notification sound can be clearly heard due to the directivity of the speaker 3208.

While the pedestrian is positioned in front of the point A side speaker and in front of the B point side speaker, the pedestrian continuously hears the notification sound with sufficient intensity.

When the pedestrian approaches the point B side speaker, most of the intensity of the notification sound is brought from the point B side speaker. Therefore, when the pedestrian reaches the rear of the point B side speaker, the intensity of the notification sound starts to be attenuated quickly.

As described above, the direction and intensity of the sound which is played toward the sidewalk by the notification speaker 3208 are set, so that the pedestrian can know that the current place is in the railroad crossing by hearing.

### <When Train Is Passing>

Now, when the train approaches the railroad crossing, the alarm speaker 3111 first emits the alarm sound, and then the railroad crossing gate 3104 is closed. Note that, in the case of our country, a standard time from the start of the alarm sound to the end of a shutdown operation is set to 15 seconds, and a standard time from the end of a shutdown operation to the arrival of the train is set to 20 seconds.

When the pedestrian is present in the railroad crossing, the pedestrian hears both of the notification sound emitted by the notification speaker 3208 and the alarm sound emitted by the alarm speaker 3111. The alarm has begun to sound, and the pedestrian must immediately leave the railroad crossing, but when the notification sound cannot be heard because of the alarm sound, the visually impaired person cannot know whether or not the current place is in the railroad crossing. Therefore, the notification sound needs to be a sound that can be heard and known at the same time as the alarm sound. For example, it is preferable to adopt a sound or a voice guidance having a different pitch (pitch of sound) or rhythm from the alarm sound as the notification sound.

The first embodiment of the dangerous area notification apparatus has been described above.

Note that, in the above description, the notification speakers 3208 are placed at both ends of the sidewalk 3103 (both on the point A side and the point B side), and covers one sidewalk by two speakers, but the notification speaker 3208 may be placed only on one side of the point A side or the point B side by letting the notification sound area be a size enough to cover one whole sidewalk.

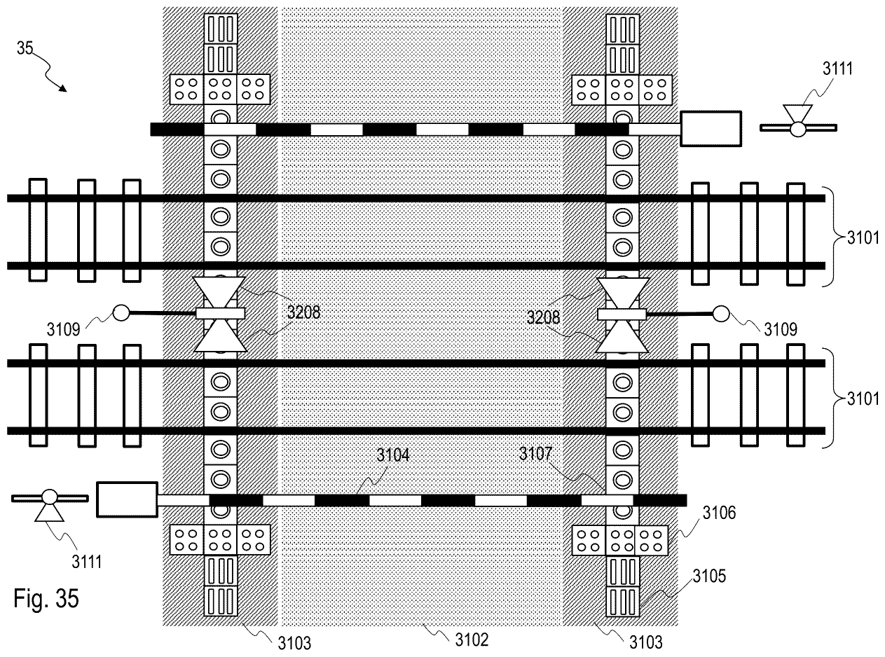

In addition, in the above description, a case where the notification speaker 3208 is installed at the single track is illustrated, but in the case of the double track, the notification speaker may be installed between railroad tracks and play the notification sound toward the outside of the railroad crossing. FIG. 35 shows an example in which the notification speaker 3208 is installed at the railroad crossing 35 of the double track.

Note that, in FIG. 35, the notification speaker 3208 may be further placed outside the railroad track, and the two speakers may cover one railroad track as the first embodiment.

In addition, in FIG. 32 and FIG. 34, an example in which the notification speaker 3208 is mounted on the dedicated support column 3109 is shown, but the notification speaker 3208 may be mounted on an existing railroad crossing facility, for example, the railroad crossing alarm sign 3112.

### Second Embodiment

In the first embodiment, the speaker having a certain degree of directivity is used as the notification speaker 3208. However, it is difficult for the cone type speaker and the horn type speaker to accurately form an acoustic area (danger notification area) in which it is possible to judge whether or not the location is in the dangerous area only by hearing. Therefore, in the second embodiment, a speaker that can locally play a sound is used instead of the notification speaker 3208 in order to accurately form the danger notification area.

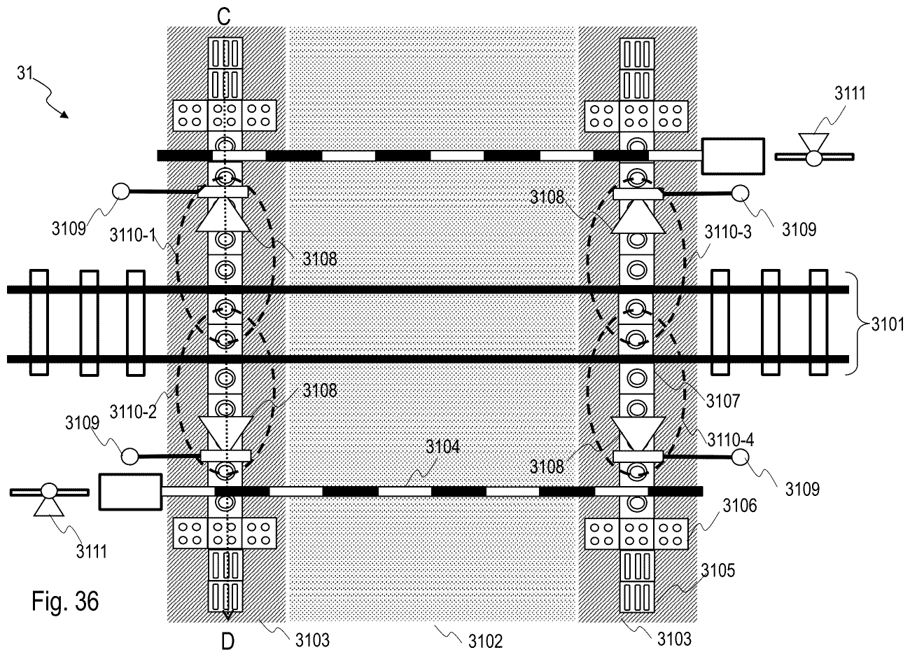

FIG. 36 is an example in which the dangerous area notification apparatus according to the second embodiment is applied to the railroad crossing 31.
3108 denotes a speaker that can locally play the sound. Here, the local play means that the sound pressure of the playback sound is controlled so as to be higher in the desired spatial area rather than the outside of said area.

Hereinafter, a target area for increasing the playback sound pressure is referred to as a localized sound area, a sound controlled so as to be locally played in the localized sound area is referred to as a localized sound, and an apparatus that can locally play a sound is referred to as a localized sound speaker.

As the localized sound speaker, it is preferable to use a speaker having a configuration described later in the present specification. As the localized sound speaker, in addition, a speaker for realizing local reproduction by combining a plurality of speakers (reference document 1: WO 2021/192166) may be used, or a parametric speaker for modulating ultrasonic waves with audible band sound waves (reference document 2: W. Gang et al., “A review of parametric acoustic array in air”, Applied Acoustics, Vol. 73, pp. 1211-1219, 2012) may be used.

In the second embodiment, the areas 3110-1 to 3110-4 in FIG. 36 are localized sound areas by the localized sound speaker 3108.
3109 denotes the support column for supporting the localized sound speaker 3108 in the air. As illustrated in FIG. 33, the localized sound speaker 3108 is placed at a height which does not interfere with the passage of an automatic person or a pedestrian.

Referring to FIG. 36, operations of a dangerous area notification apparatus will be described.

### <When No Train is Passing>

The localized sound is played from the localized sound speaker 3108. The localized sound may be a voice announcement or a non-voice such as a buzzer sound or a chime sound.

When the pedestrian moves from the point C to the point D, the pedestrian experiences the following. Note that it is assumed that the railroad crossing gate is not closed. In addition, it is assumed that the same localized sound is played from the four localized sound speakers 3108.

At the point C, the localized sound is not heard by the pedestrian. When approaching the localized sound area 3110-1, the weak localized sound is heard by the pedestrian, and the localized sound gradually increases as approaching the localized sound area 3110-1.

When the pedestrian reaches the vicinity of the point C side boundary of the localized sound area 3110-1, the intensifying rate of the localized sound heard by the pedestrian increases, and when the pedestrian enters the localized sound area 3110-1, the localized sound is clearly heard.

In the example in FIG. 36, since the localized sound areas 3110-1 and 3110-2 overlap each other, the pedestrian continuously hears the localized sound over the localized sound areas 3110-1 to 3110-2.

When the pedestrian approaches the point D side boundary of the localized sound area 3110-2, the localized sound starts to suddenly weaken, and when the pedestrian comes out of the localized sound area 3110-2, the localized sound is so small that it cannot be detected without attention. The localized sound gradually becomes even smaller as moving away from the localized sound area 3110-2 and becomes unheard at the point D.

By this, the localized sound area of the localized sound speaker is set in the railroad crossing, so that the pedestrian can know that the current place is in the railroad crossing by hearing.

### <When Train is Passing>

Similarly to the description of the first embodiment, when the localized sound cannot be heard because of the alarm sound, the visually impaired person cannot know whether or not the current place is in the railroad crossing. Therefore, the localized sound needs to be a sound that can be heard and known at the same time as the alarm sound. For example, it is preferable to adopt a sound or a voice guidance having a different pitch (pitch of sound) or rhythm from the alarm sound as the localized sound.

The second embodiment of the dangerous area notification apparatus has been described above.

Note that, in the above description, localized sound speakers 3108 are placed at both ends of the sidewalk 3103 (both on the point C side and the point D side), and one sidewalk is covered with two localized sound areas, but the localized sound speaker is set only on one side of the point C or the point D side by letting the localized sound area be a size to cover one whole sidewalk.

In addition, in FIG. 36, an example in which the localized sound speaker 3108 is mounted on the dedicated support column 3109, but the localized sound speaker 3108 may be mounted on the existing railroad crossing facility, for example, the railroad crossing alarm sign 3112.

### Third Embodiment

In the second embodiment, the localized sound speaker is placed outside the railroad track with respect to the sidewalk crossing the single track. In a third embodiment, the localized sound speaker is placed inside the railroad track with respect to the sidewalk crossing the double track.

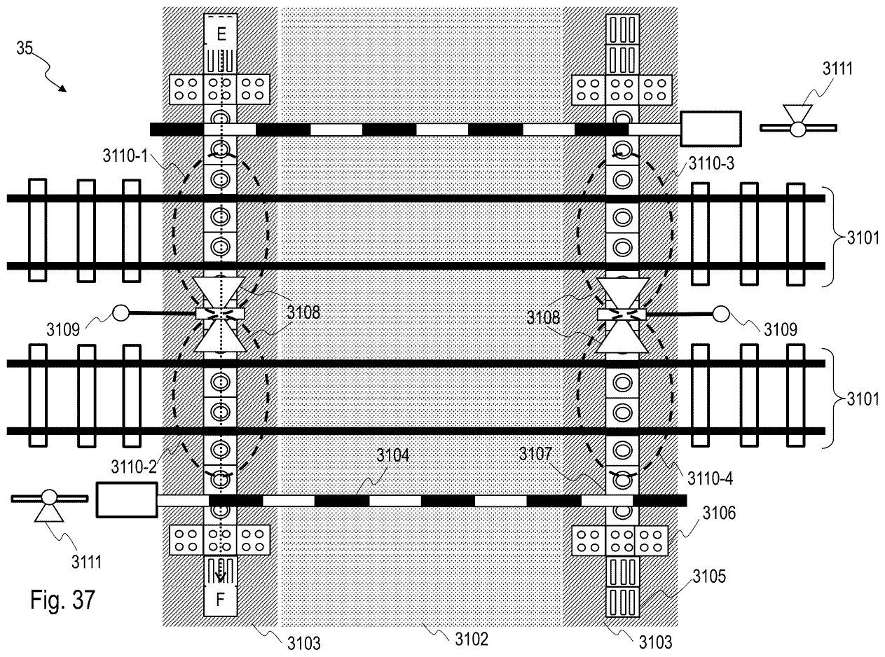

Referring to FIG. 37, the third embodiment of the dangerous area notification apparatus will be described. A difference from the second embodiment is the position of the localized sound speaker 3108. The support columns 3109 are placed between the railroad tracks, and the localized sound areas 3110-1 to 3110-4 cover the sidewalk on the railroad tracks respectively by one.

When the train is not passing, the localized sound of the pedestrian moving from the point E to the point F is the same as that in the second embodiment.

It is similar to the second embodiment in that a sound that can be heard at the same time as the alarm sound is adopted as the localized sound in a case that the train passes.

The third embodiment of the dangerous area notification apparatus has been described above.

### Fourth Embodiment

In the second embodiment and third embodiment, the localized sound area is set in the sidewalk in the railroad crossing, and the localized sound is heard to know that the current place is inside the railroad crossing.

By the way, the sidewalk of the railroad crossing has a risk of contact with an automobile when stepping off to the road side, and has a risk of falling down due to a step or a rail when stepping off to the railroad track side. Therefore, in a fourth embodiment, the localized sound area is set so that it can be known that the pedestrian has been off the sidewalk in the railroad crossing.

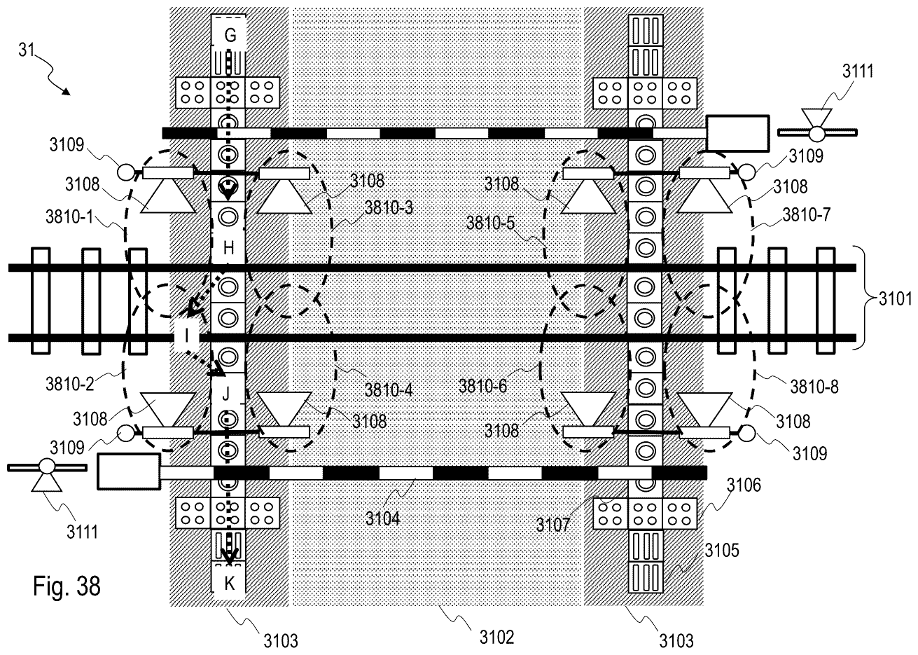

Referring to FIG. 38, the fourth embodiment of the dangerous area notification apparatus will be described. The case where the railroad track is the single track is used as an example.

Although it is similar to the second embodiment that the localized sound speakers are placed so as to sandwich the railroad tracks in the fourth embodiment, two localized sound speakers 3108 are placed for each support column 3109, the localized sound areas 3810-1 and 3810-2 cover the railroad track side with respect to the tactile paving and the localized areas 3810-3 and 3810-4 cover the roadway side with respect to the tactile paving. The same is applied to the localized sound areas 3810-5 to 3810-8. At this time, the localized sound area is set so as not to cover the tactile paving.

When the pedestrian (visually impaired person) moving from the point G to the point K is off the tactile paving and then returned to the tactile paving, the visually impaired person experiences the following.

The visually impaired person travels along the tactile paving from the point G to the point H. Since the point H is outside the localized sound area, the visually impaired person does not hear the localized sound (dangerous area notification).

It is assumed that the visually impaired person is off the tactile paving in the railroad crossing and moves to the point I. Since the point I is in the localized sound area 3810-2, the visually impaired person hears the localized sound.

The localized sound in this case is set to “You are in the railroad crossing. You are off the tactile paving toward the railroad track side.”, for example. Then, the visually impaired person can know that the tactile paving is no longer under the feet, but that the current place is in the railroad crossing and that the tactile paving is probably present on his/her approximate left side since being off toward the railroad track side.

The same localized sound as the localized sound area 3810-2 is played to the localized sound areas 3810-1, 3810-7, and 3810-8. The localized sound played to the localized sound areas 3810-3, 3810-4, 3810-5, and 3810-6 on the roadway side is set to “You are in the railroad crossing. You are off the tactile paving toward the roadway side.”, for example.

The fourth embodiment of the dangerous area notification apparatus has been described above.

Note that, in the fourth embodiment, the localized sound speakers 3108 are placed so as to sandwich the railroad track, and the four localized sound areas (for example, 3810-1 to 3810-4) cover the outside of one sidewalk, but the localized sound speakers may be placed either of the point G side or the point K side, and the two localized sound areas may cover the outside of one sidewalk. Alternatively, one localized sound speaker 3108 is placed on the point G side and one localized sound speaker 3108 is placed on the point K side, and the speaker on the point G side may cover the railroad track side of the tactile paving in the railroad crossing and the point K side may cover the roadway side.

In addition, although the fourth embodiment has been described with reference to the case of the single track as an example, similarly to the third embodiment, the localized sound speaker may be placed inside the railroad track, and the four speakers may cover the outside of the tactile paving of one sidewalk crossing the double track.

Further, in the fourth embodiment, the localized sound area is set so as to sandwich the sidewalk “in the railroad crossing”, but the localized sound area may be set so as to cover, for example, the tactile paving 3106 “in front of the railroad crossing”. If so, the pedestrian can know, by hearing, that this area ahead is in the railroad crossing, or he/she has completed passing the railroad crossing safely.

Note that instead of the localized sound speaker and the localized sound area of the fourth embodiment, the notification speaker and the notification sound area of the first embodiment may be used. That is, the notification area may be set in the peripheral area of the sidewalk (or tactile paving) in the rail road crossing such as areas sandwiching the sidewalk (or tactile paving) in the rail road crossing or areas in front of the railroad crossing like those of the fourth embodiment.

### [Modification Example of Dangerous Area Notification Apparatus]

### <Timing of Playing Notification Sound/Localized Sound>

In the above embodiments, the notification sound/localized sound is always played, but the notification sound/localized sound may be played intermittently or may be played only when the pedestrian passes.

A human sensor for sensing the pedestrian can be used as a trigger for playing the notification sound/localized sound only when the pedestrian passes. Alternatively, a pressure sensor may be provided on the tactile paving in the railroad crossing, and the notification sound/localized sound may be played by detecting that the pedestrian steps on the tactile paving.

The notification sound/localized sound may be played only when the visually impaired person passes. For example, a push button for the visually impaired person may be provided in front of the railroad crossing, and when the visually impaired person pushes the button, the notification sound/localized sound may be played.

Alternatively, the visually impaired person may be automatically detected. For example, object recognition may be performed on a white stick carried by the pedestrian and the notification sound/localized sound may be played, or a passive tag may be placed on the white stick, the passive tag is detected by the railroad crossing facility, and the notification sound/localized sound may be played.

### <Contents of Notification Sound/Localized Sound>

In addition, the contents of the notification sound/localized sound may be changed when the train is not passing (when the alarm speaker is not operated) or when the train is approaching (when the alarm speaker is operated).

For example, when the alarm speaker is not operated, the notification sound/localized sound is a music recognized by anyone related to the railroad, and in a scene where the alarm speaker is operated, an announcing of a time until the railroad crossing gate is closed and a time until the train reaches is set to the contents together with a sound for informing danger, such as an emergency earthquake prompt report.

### <Safety Area Notification>

In the above-described embodiments, the visually impaired person confirms that the person has left the dangerous area when he/she becomes unable to hear the dangerous area notification (localized sound). The visually impaired person who recognized alarm sound and has left the railroad crossing may be concerned that simply “not hearing” the dangerous area notification does not mean that the person has truly escaped to a safe place.

Therefore, the localized sound area may be set to the outside of the railroad crossing and immediately in front of the railroad crossing gate, and the localized sound (safety area notification) having the contents of “You are out of the railroad crossing” may be played.

The safety area notification may be played, for example, when the alarm speaker is operated. Then, while suppressing unnecessary sound presentation to the outside of the railroad crossing, the visually impaired person can be assured that the person has escaped from the dangerous area in emergency.

### <Functional Block Diagram of Modification Example>

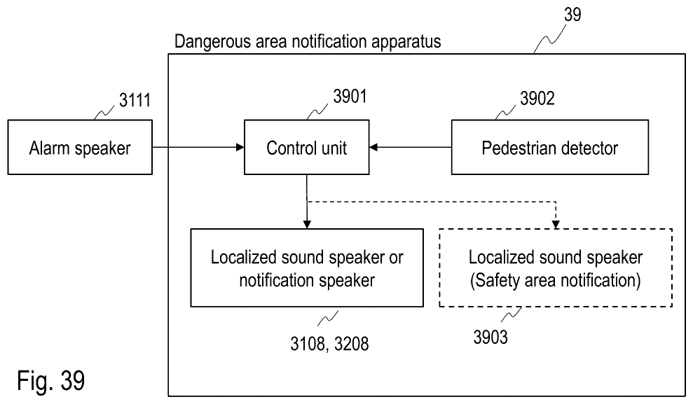

FIG. 39 is a functional block diagram of the dangerous area notification apparatus for controlling the notification sound/localized sound as above. The dangerous area notification apparatus 39 includes a control unit 3901, a pedestrian detection unit 3902, a localized sound speaker 3108, or a notification speaker 3208. As necessary, a safety area notification localized sound speaker 3903 is further included. The alarm speaker 3111 is an existing speaker.

The pedestrian detection unit 3902 detects the presence or absence of the pedestrian and whether or not the pedestrian is the visually impaired person, and outputs a result to the control unit 3901.

The alarm speaker 3111 outputs information on whether or not the alarm sound is being emitted to the control unit 3901. The control unit 3901 controls the localized sound emitted by the localized sound speaker 3108 or the notification sound emitted by the notification speaker 3208 on the basis of input from the pedestrian detection unit 3902 and the alarm speaker 3111. Further, the localized sound generated by the safety area notification localized sound speaker 3903 is controlled as necessary.

The modification example of the first embodiment to the fourth embodiment have been described above.

### Fifth Embodiment

Although the dangerous area notification apparatus in which the railroad crossing is set to the dangerous area has been described in the first embodiment to the fourth embodiment, a dangerous area notification apparatus in which a crosswalk is set to the dangerous area will be described in a fifth embodiment.

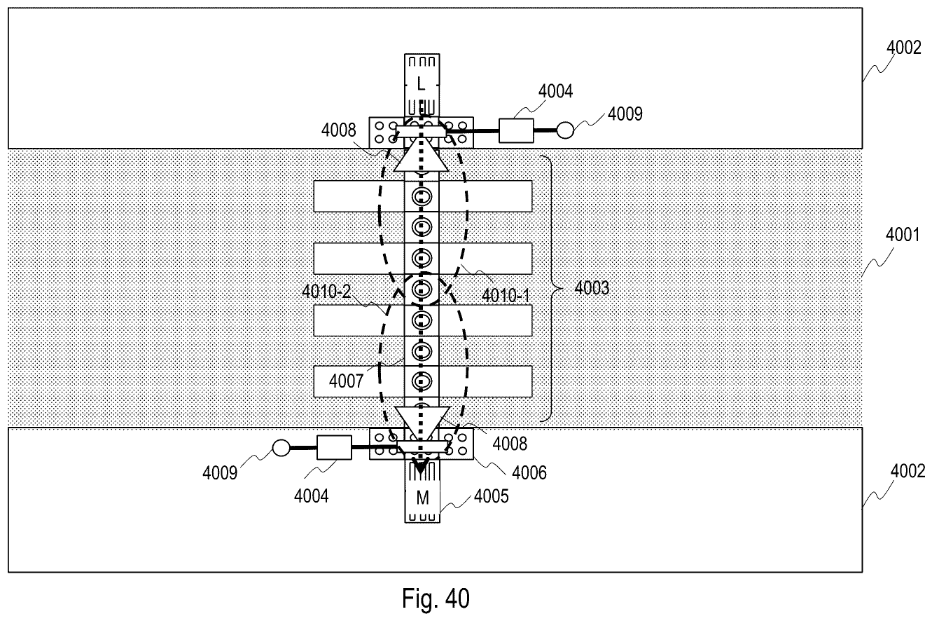

FIG. 40 is an example in which the dangerous area notification apparatus is applied to the crosswalk. 4001 denotes the roadway, 4002 denotes the sidewalk, 4003 denotes the crosswalk crossing the roadway, and 4004 denotes a pedestrian traffic signal.

Tactile paving 4005, 4006, and 4007 having different tactile shapes are installed on the sidewalk and the crosswalk.

The tactile paving 4005 is provided with tactile shape indicating that this area ahead is the crosswalk, for example.

The tactile paving 4006 is provided with tactile shape indicating that this area ahead is the roadway (crosswalk), for example.

The tactile paving 4007 is provided with tactile shape indicating that the location is in the roadway (on the crosswalk), for example. Although FIG. 40 is an example in which the tactile paving 4007 is placed at the center of the crosswalk, the positional relationship between the crosswalk and the tactile paving is not limited to this, and the tactile paving may be placed at the end or near the end of the crosswalk. The tactile paving 4005 and 4006 are placed in accordance with the position of the tactile paving 4007.
4008 denotes the speaker that can locally play the sound.

In the fifth embodiment, the areas 4010-1 and 4010-2 are localized sound areas by the localized sound speaker 4008.
4009 denotes the support column for supporting the localized sound speaker 4008 in the air. The localized sound speaker 4008 is placed at a height which does not interfere with the passage of the pedestrian.

Now, the localized sound is played from the localized sound speaker 4008 placed as above. When the pedestrian moves from the point L to the point M, the pedestrian experiences the following. Note that it is assumed that the same localized sound is played from the two localized sound speakers 4008.

At the point L, the localized sound is not heard by the pedestrian. When approaching the localized sound area 4010-1, a weak localized sound is heard by the pedestrian, and the localized sound gradually increases as approaching the localized sound area 4010-1.

When the pedestrian reaches the vicinity of the point L side boundary of the localized sound area 4010-1, the intensifying rate of the localized sound heard by the pedestrian increases, and when the pedestrian enters the localized sound area 4010-1, the localized sound is clearly heard.

In the example in FIG. 40, since the localized sound areas 4010-1 and 4010-2 overlap each other, the pedestrian continuously hears the localized sound over the localized sound areas 4010-1 to 4010-2.

When the pedestrian approaches the point M side boundary of the localized sound area 4010-2, the localized sound starts to suddenly weaken, and when the pedestrian comes out of the localized sound area 4010-2, the localized sound is so small that it cannot be detected without attention. The localized sound gradually becomes even smaller as moving away from the localized sound area 4010-2 and becomes unheard at point M.

By this, when the localized sound area of the localized sound speaker 4008 is set on the crosswalk, the pedestrian can know that the current place is on the roadway by hearing.

The fifth embodiment of the dangerous area notification apparatus has been described above.

Note that, in the above description, the localized sound speakers 4008 are placed so as to sandwich the roadway 4001 (both on the point L side and the point M side), and the tactile paving of the crosswalk is covered with two localized sound areas, but the localized sound speaker may be placed only on one side of the L point side or the M point side by letting the single localized sound area be a size to cover the whole tactile paving of the crosswalk.

In addition, in the example in FIG. 40, the support column 4009 supports both the pedestrian traffic signal 4004 and the localized sound speaker 4008, but the support column for the pedestrian traffic signal and the support column for the localized sound speaker may be installed respectively.

In addition, when the traffic signal is an acoustic type traffic signal (a traffic signal having an apparatus for emitting a guide sound in order to inform the visually impaired person that the traffic signal is green now), the localized sound needs to be a sound that can be heard and known at the same time as the guide sound, similarly to the case of the alarm sound of the railroad crossing. For example, it is preferable to adopt a sound having a pitch (pitch of sound) or rhythm different from that of the guide sound or a voice guide as the localized sound.

### [Supplement to Embodiment of Dangerous Area Notification Apparatus]

In the above description, the operations of the notification speaker 3208 and the localized sound speakers 3108 and 4008 are described together with the arrangement of the tactile paving, but it is to be noted that the presence of the tactile paving is not essential for the dangerous area notification by the notification speaker and the localized sound speaker to be effective. The present disclosed technology does not merely support the function of the tactile paving, but can be an alternative to the tactile paving and allows the visually impaired person to easily know that the current place is the dangerous area to remain in such as a railroad crossing or crosswalk, and that he/she has escaped from the dangerous area.

### [Localized Sound Speaker]

Hereinafter, a speaker suitable as the localized sound speaker of the dangerous area notification apparatus will be described.

### [First Embodiment of Localized Sound Speaker]

First, a first embodiment of the localized sound speaker will be described.

### <Configuration>

An acoustic signal output apparatus 10 of the present embodiment is an acoustic hearing apparatus (for example, earphone of open ear type (open type), headphone, speaker of stationary type, speaker of embedded type, or the like) that is mounted without sealing a user's ear canal. As illustrated in FIG. 1 to FIG. 4, the acoustic signal output apparatus 10 of the present embodiment has a concave type (for example, parabolic shape type) reflector 13 having a paraboloid of revolution or a surface approximate to a paraboloid of revolution inside, driver units 11 and 15 (in other words, a speaker driver or a driver) for converting an output signal (electric signal representing an acoustic signal) outputted from a reproducing apparatus into the acoustic signal and outputting it, a housing 16 for storing driver unit 15 inside, and a support part 14 for placing the driver unit 11 inside the reflector 13.

### <Driver Unit 11 (First Driver Unit)>

In the present embodiment, a frequency band of the acoustic signal (reproduced acoustic signal) to be reproduced is divided into a high frequency band and a low frequency band, and the driver unit 11 emits the acoustic signal on the high frequency band side among the reproduced acoustic signals. That is, the driver unit 11 handles mainly the acoustic signal of a high band among the reproduced acoustic signals. The output signal outputted from the reproducing apparatus is separated into a high frequency band signal on the high frequency side and a low frequency band signal on the lower frequency side than that, and the high frequency band signal separated in this way is inputted to the driver unit 11. Note that the frequency bands in which the levels of the high frequency band signal and the low frequency band signal are equal to or higher than a predetermined value may be overlapped with each other or may not be overlapped with each other. The driver unit 11 is a device (a device having a speaker function) that emits (plays) an acoustic signal AC1 (first acoustic signal) on the basis of the inputted high frequency band signal to first side (D1 direction side) and emits an acoustic signal AC2 (second acoustic signal) which is an inverse phase signal (phase inversed signal) of the acoustic signal AC1 or an approximate signal of the inverse phase signal to the other side (D2 direction side). That is, an acoustic signal emitted from the driver unit 11 to first side (D1 direction side) is called the acoustic signal AC1 (first acoustic signal), and an acoustic signal emitted from the driver unit 11 to the other side (D2 direction side) is called the acoustic signal AC2 (second acoustic signal). For example, the driver unit 11 is placed on an axis line A1 (axis) or in the vicinity of the axis line A1 (axis) extending along the D1 direction, and the acoustic signals AC1 and AC2 are emitted along the axis line A1 (axis). For example, the driver unit 11 includes a diaphragm 113 which emits the acoustic signal AC1 to the D1 direction side from one surface 113a by vibration and emits the acoustic signal AC2 to the D2 direction side from the other surface 113b by this vibration (FIG. 1). For example, the diaphragm 113 is placed on the axis line A1 (axis) or in the vicinity of the axis line A1 (axis). The driver unit 11 in this example emits the acoustic signal AC1 from a surface 111 to the D1 direction side (first side) by the vibration of the diaphragm 113 on the basis of the inputted high frequency band signal and emits the acoustic signal AC2 which is the inverse phase signal or the approximate signal of the inverse phase signal of the acoustic signal AC1 from a surface 112 to the D2 direction side (the other side). That is, the acoustic signal AC2 is secondarily emitted as the acoustic signal AC1 is emitted. Note that although the D2 direction (the other side) is, for example, the inverse direction to the D1 direction (first side), the D2 direction need not be strictly the inverse direction to the D1 direction, and the D2 direction may be different from the D1 direction. The relationship between the first side (D1 direction) and the other side (D2 direction) depends on a system and shape of the driver unit 11. In addition, depending on the system and shape of the driver unit 11, the acoustic signal AC2 may become the strictly inverse phase signal of the acoustic signal AC1 and the acoustic signal AC2 may become the approximate signal of the inverse phase signal of the acoustic signal AC1. For example, the approximate signal of the inverse phase signal of the acoustic signal AC1 may be (1) a signal obtained by shifting the phase of the inverse phase signal of the acoustic signal AC1, (2) a signal obtained by changing (amplifying or attenuating) amplitude of the inverse phase signal of the acoustic signal AC1, or (3) a signal obtained by shifting the phase of the inverse phase signal of the acoustic signal AC1 and changing its amplitude. A phase difference between the inverse phase signal of the acoustic signal AC1 and its approximate signal is preferably 51% or less of one period of the inverse phase signal of the acoustic signal AC1. Examples of 51% are 1%, 3%, 5%, 10%, 20%, and the like. In addition, the difference between the amplitude of the inverse phase signal of the acoustic signal AC1 and the amplitude of its approximate signal is preferably 52% or less of the amplitude of the inverse phase signal of the acoustic signal AC1. Examples of 52% are 1%, 3%, 5%, 10%, 20%, and the like. Note that a dynamic type, a balanced armature type, a hybrid type of the dynamic type and the balanced armature type, a condenser type, or the like exemplify the mechanism of the driver unit 11. In addition, the shapes of the driver unit 11 and the diaphragm 113 have no limitations. In the present embodiment, in order to simplify the description, an example will be shown in which an outer shape of the driver unit 11 is an approximately cylindrical shape with both end surfaces, and the diaphragm 113 is an approximately disc shape, but the disclosed technology is not limited thereto. For example, the outer shape of the driver unit 11 may be a rectangular parallelepiped shape, and the diaphragm 113 may be a dome shape. In addition, examples of the acoustic signal are sounds such as music, vocal sound, sound effects, and environmental sounds.

### <Driver Unit 15 (Second Driver Unit)>

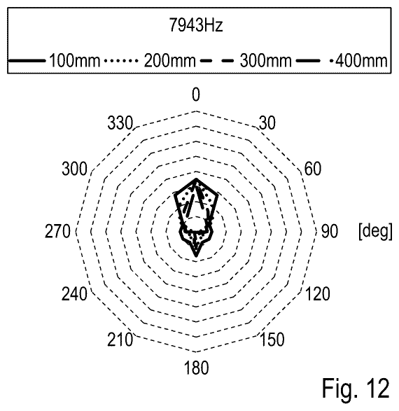

The driver unit 15 of the present embodiment is placed on the D2 direction side of the driver unit 11. The driver unit 15 is larger in size than the driver unit 11, and emits the acoustic signal on the low frequency band side among the above-mentioned reproduced acoustic signals. That is, the driver unit 15 handles mainly the acoustic signal of a low band among the reproduced acoustic signals. By this, sound pressure of the low band can be obtained as compared with the case where only the driver unit 11 is used. As mentioned above, the low frequency band signal separated from the output signal is inputted to the driver unit 15 and the driver unit 15 is a device (device having a speaker function) that emits (plays) an acoustic signal AC3 (third acoustic signal) on the basis of the inputted low frequency band signal to first side (D1 direction side) and emits an acoustic signal AC4 (fourth acoustic signal) which is an inverse phase signal (phase inversed signal) or an approximate signal of the inverse phase signal of the acoustic signal AC3 to the other side (D2 direction side). That is, the acoustic signal emitted from the driver unit 15 to first side (D1 direction side) is called the acoustic signal AC3 (third acoustic signal), and the acoustic signal emitted from the driver unit 15 to the other side (D2 direction side) is called the acoustic signal AC4 (fourth acoustic signal). For example, the driver unit 15 is placed on the axis line A1 (axis) or in the vicinity of the axis line A1 (axis), and the acoustic signals AC3 and AC4 are emitted along the axis line A1 (axis). The driver unit 15 includes a diaphragm 153 (second diaphragm) which emits the acoustic signal AC3 (third acoustic signal) from one surface 153a to the D1 direction side (first side) by the vibration and emits the acoustic signal AC4 (fourth acoustic signal) from the other surface 153b to the D2 direction side (the other side) by this vibration (FIG. 12). For example, the diaphragm 153 is placed on the axis line A1 (axis) or in the vicinity of the axis line A1 (axis). The driver unit 15 of this example emits the acoustic signal AC3 from the surface 151 on first side to the D1 direction side by the vibration of the diaphragm 153 on the basis of the inputted low frequency band signal and emits the acoustic signal AC4 which is the inverse phase signal or the approximate signal of the inverse phase signal of the acoustic signal AC3 from the surface 152 on the other side to the D2 direction side. That is, the acoustic signal AC4 is secondarily emitted as the acoustic signal AC3 is emitted. The acoustic signal AC3 is an in-phase signal or an approximate signal of the in-phase signal of the acoustic signal AC1, and the acoustic signal AC4 is an in-phase signal or an approximate signal of the in-phase signal of the acoustic signal AC2. Note that, depending on the system and shape of the driver unit 15, the acoustic signal AC4 may strictly be the inverse phase signal of the acoustic signal AC3 or the acoustic signal AC4 may be the approximate signal of the inverse phase signal of the acoustic signal AC3. For example, the approximate signal of the inverse phase signal of the acoustic signal AC3 may be (1) a signal obtained by shifting the phase of the inverse phase signal of the acoustic signal AC3, (2) a signal obtained by changing (amplifying or attenuating) the amplitude of the inverse phase signal of the acoustic signal AC3, or (3) a signal obtained by shifting the phase of the inverse phase signal of the acoustic signal AC3 and changing its amplitude. The phase difference between the inverse phase signal of the acoustic signal AC3 and its approximate signal is preferably δ3% or less of one period of the inverse phase signal of the acoustic signal AC3. Examples of δ3% are 1%, 3%, 5%, 10%, 20%, and the like. In addition, the difference between the amplitude of the inverse phase signal of the acoustic signal AC3 and the amplitude of its approximate signal is preferably δ4% or less of the amplitude of the inverse phase signal of the acoustic signal AC3. Examples of δ4% are 1%, 3%, 5%, 10%, 20%, and the like. Note that a dynamic type, a balanced armature type, a hybrid type of the dynamic type and the balanced armature type, a condenser type, and the like exemplify the mechanism of the driver unit 15. In addition, the shapes of the driver unit 15 and the diaphragm 153 have no limitation. In the present embodiment, in order to simplify the description, an example will be shown in which an outer shape of the driver unit 15 is an approximately cylindrical shape with both end surfaces, and the diaphragm 153 is an approximately disc shape, but the disclosed technology is not limited thereto. For example, the outer shape of the driver unit 15 may be a rectangular parallelepiped shape, and the diaphragm 153 may be a dome shape.

As mentioned above, the driver unit 15 is larger in size than the driver unit 11. For example, when a diameter (diameter in a direction orthogonal to the D1 direction and/or the D2 direction) of the driver unit 11 is set to S11, and a diameter (diameter in a direction orthogonal to the D1 direction and/or the D2 direction) of the driver unit 15 is set to S21, S21>S11 is satisfied. S21 may be twice or more of S11, for example, S11 is 12 mm, and S21 is 35 mm. In addition, for example, when the diameter (diameter in a direction orthogonal to the D1 direction and/or the D2 direction) of the diaphragm 113 is set to S12 and the diameter (diameter in a direction orthogonal to the D1 direction and/or the D2 direction) of the diaphragm 153 is set to S22, S22>S12 is satisfied. S22 may be twice or more of S12, for example, S12 is 10 mm, and S22 is 30 mm. That is, the diameter of the diaphragm 153 (second diaphragm) is larger than the diameter of the diaphragm 113 (first diaphragm).

### <Reflector 13 and Support Part 14>

The reflector 13 is a concave type structure having the paraboloid of revolution or the surface approximate to the paraboloid of revolution inside. That is, at least a part of the inner wall surface 131 of the reflector 13 is the paraboloid of revolution or the surface approximate to the paraboloid of revolution. The paraboloid of revolution has, for example, a shape in which the parabola is rotated around the axis line A1 (specific axis). The whole inner wall surface 131 may be the paraboloid of revolution or the surface approximate to the paraboloid of revolution, or only a part of the inner wall surface 131 (for example, only the inner wall surface 131 on the bottom part 131a side of the reflector 13 or only the inner wall surface 131 on the tip part 131c side) may be the paraboloid of revolution or the surface approximate to the paraboloid of revolution.

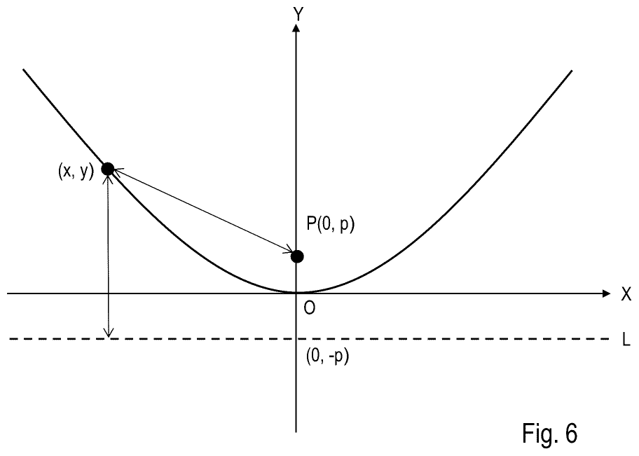

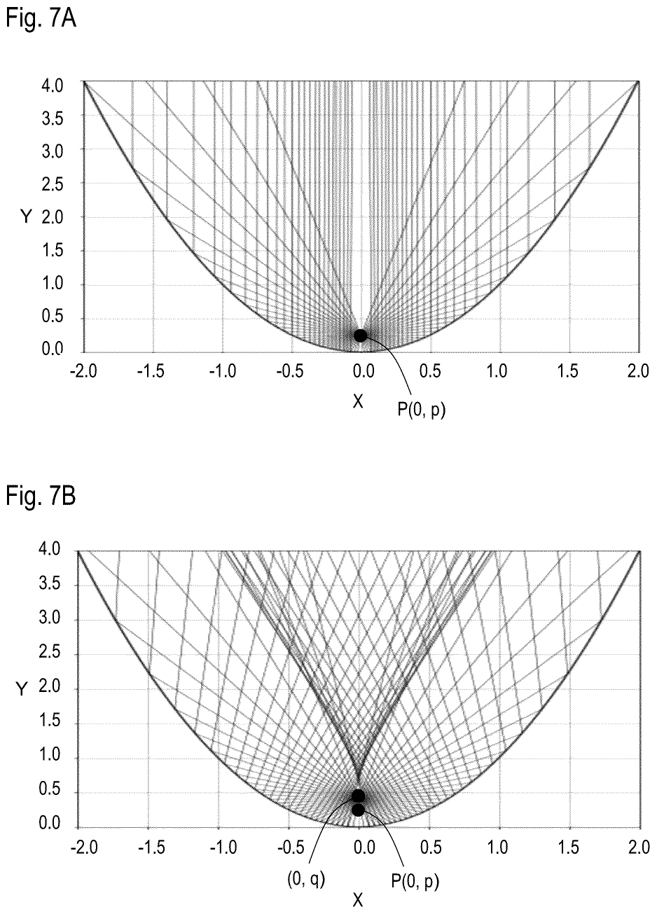

The driver unit 11 is placed inside the reflector 13. The driver unit 11 is fixed to the inner wall surface 131 of the reflector 13 through the support part 14. In the present embodiment, one surface 111 of the driver unit 11 placed inside the reflector 13 is directed to the open end 130 side (D1 direction side) of the reflector 13, and the surface 112 on the other side is directed to the bottom part 131a side (D2 direction side) of the reflector 13. The driver unit 11 (first driver unit) emits the acoustic signal AC1 (first acoustic signal) to the D1 direction side (first side) of the driver unit 11 and emits the acoustic signal AC2 (second acoustic signal) to the D2 direction side (the other side) of the driver unit 11. The acoustic signal AC1 (reproduced acoustic signal) emitted from the driver unit 11 is emitted outward from the open end 130 on the D1 direction side of the reflector 13. Here, a part of the acoustic signal AC1 is directly emitted from the driver unit 11 to the D1 direction side of the reflector 13. In addition, at least another part of the acoustic signal AC1 is reflected by the inner wall surface 131 of the reflector 13, and then emitted from the open end 130 to the D1 direction side. Further, at least a part of the acoustic signal AC2 is reflected by the inner wall surface 131 of the reflector 13, and then emitted from the open end 130 to the D1 direction side. A user located on the D1 direction side can hear the acoustic signal AC1 emitted from the open end 130 of the reflector 13. In this case, the reflector 13 suppresses sound leakage of the acoustic signal AC1 to the rear surface 132 side of the reflector 13. In addition, the acoustic signal AC2 is the inverse phase signal or the approximate signal of the inverse phase signal of the acoustic signal AC1. Therefore, at a specific position (for example, a position behind the user) on the D1 direction side other than the presence of the user, a part of the acoustic signal AC1 and a part of the acoustic signal AC2 cancel each other out, and sound leakage of the acoustic signal AC1 is suppressed. Note that it is desirable that the driver unit 11 is placed on the axis line A1, and for example, it is desirable that the diaphragm 113 is placed on the axis line A1. More preferably, it is desirable that the center of the diaphragm 113 or its vicinity is placed on the axis line A1. In other words, it is desirable that the diaphragm 113 is placed at or in the vicinity of the center of the above-mentioned paraboloid of revolution. As a result, the sound pressure of the acoustic signal AC1 emitted from the open end 130 becomes axis-symmetric or approximately axis-symmetric with respect to the axis line A1. In addition, more preferably, it is desirable that the driver unit 11 is placed at the focal point or in the vicinity of the focal point of the paraboloid of revolution. In this case, the directivity of the acoustic signal AC1 emitted from the open end 130 becomes high. This will be described in detail. As illustrated in FIG. 6, in X-Y coordinates, a point on a parabola constituting the paraboloid of revolution is defined as (x, y), the focal point of the paraboloid of revolution is defined as P (0, p), and a directrix parallel to the X axis passing through the point (0, −p) is defined as L: y=−p. Where, p≠0 is satisfied. In this case, a set of points (x, y) having equal distances from the focal point P (0, p) and the directrix L: y=−p satisfies x²=4py. As illustrated in FIG. 7A, when the driver unit 11 is placed at the focal point P (0, p) or in the vicinity of the focal point P (0, p) of the paraboloid of revolution, the center of the traveling direction of the acoustic signal AC1 emitted from the open end 130 is parallel to the Y axis (axis line A1). Therefore, when the driver unit 11 is placed at the focal point P (0, p) or in the vicinity of the focal point P (0, p) of the paraboloid of revolution, the directivity of the acoustic signal AC1 emitted from the open end 130 becomes high. On the other hand, as illustrated in FIG. 7B, when the driver unit 11 is placed at the position (0, q) deviated from the focal point P (0, p) or the vicinity of the focal point P (0, p) of the paraboloid of revolution (p≠q), the center of the traveling direction of the acoustic signal AC1 emitted from the open end 130 diffuses outward with respect to the Y axis. In this case, the directivity of the acoustic signal AC1 emitted from the reflector 13 is lower than the case where the driver unit 11 is placed at the focal point P (0, p) or in the vicinity of the focal point P (0, p) of the paraboloid of revolution.

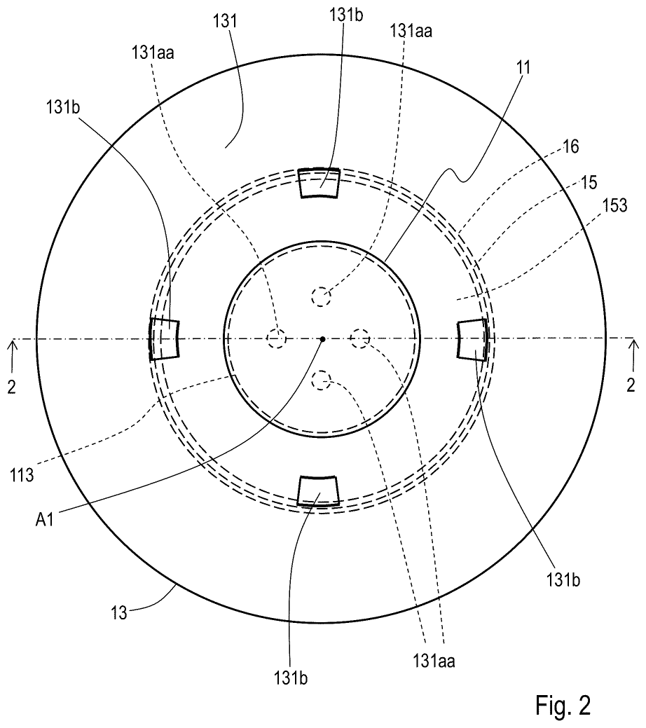

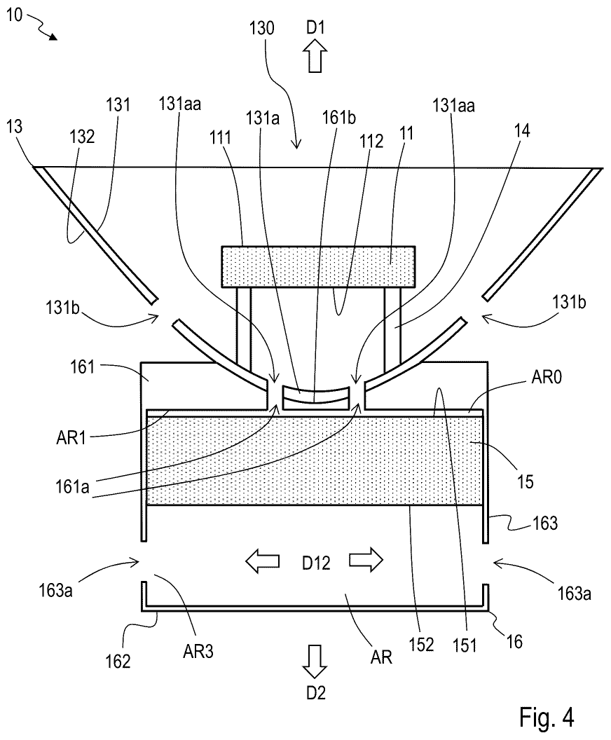

The acoustic signals AC1 and AC2 have shorter wavelengths and higher straightness as the frequency is higher. Therefore, the directivity of the high band components of the acoustic signals AC1 and AC2 emitted from the open end 130 of the reflector 13 is high, and the high band components are hardly leaked to the rear surface 132 side of the reflector 13. Here, a part of the acoustic signal AC2 is also reflected by the inner wall surface 131 of the reflector 13, and then emitted from the open end 130 to the D1 direction side. The acoustic signal AC2 is the inverse phase signal or the approximate signal of the inverse phase signal of the acoustic signal AC1. However, these high band components have short wavelengths and are difficult to cancel each other out. Therefore, the sound pressure of the high band components of the acoustic signal AC1 can be sufficiently secured on the D1 direction side. On the other hand, the middle/low band components of the acoustic signals AC1 and AC2 emitted from the open end 130 have the low directivity, and are easily leaked to the rear surface 132 side. However, the acoustic signal AC2 is the inverse phase signal or the approximate signal of the inverse phase signal of the acoustic signal AC1, and these low band components have long wavelengths and are easily cancel each other out. Therefore, even if the low band components of the acoustic signals AC1 and AC2 leak to the rear surface 132 side, they cancel each other out to suppress sound leakage. In order that the acoustic signal AC2 and the acoustic signal AC1 cancel each other out at a position where the sound leakage is to be suppressed, it is ideal that a difference between a propagation distance from first side surface 111 of the driver unit 11 to the position where the sound leakage is to be suppressed and a propagation distance from the other side surface 112 of the driver unit 11 to the position where the sound leakage is to be suppressed is an integer multiple (including the same) of the wavelength of the acoustic signals AC1 and AC2. In order to optimize this condition, the reflector 13 of the present embodiment is provided with a single or a plurality of sound holes 131b (reflector sound holes). By this, sound leakage of middle/low band components of the acoustic signals AC1 and AC2 can be suppressed. In addition, the sound hole 131b also has an effect of weakening the directivity of high band components of the acoustic signals AC1 and AC2. When the sound pressure of the high band components is too high, it may be grating to the ear, but the sound pressure of the high band components of the acoustic signals AC1 and AC2 emitted to the D1 direction side can be weakened by providing the sound holes 131b. Note that, in FIG. 2 or the like, four rectangular sound holes 131b are arranged in the reflector 13 with axis-symmetry or approximate axis-symmetry with respect to the axis line A1. However, this does not limit the disclosed technology, the sound holes 131b of circular or triangular shape may be provided, a plurality of sound holes 131b different in shape and size from each other may be provided, or the sound holes 131b may be arranged in a deviated manner at any position. For example, the sound holes 131b may be arranged in a deviated manner in a direction where sound leakage of the acoustic signal AC1 becomes a problem. In addition, as illustrated in FIG. 1, FIG. 4, or the like, it is desirable that the sound hole 131b is arranged in the D2 direction side (the other side) of the driver unit 11 (first driver unit) or in the vicinity of the D2 direction side (the other side) of the driver unit 11 (first driver unit). By this, the acoustic signal AC1 emitted from the D1 direction side of the driver unit 11 is hardly emitted from the sound hole 131b, and the acoustic signal AC2 emitted from the D2 direction side of the driver unit 11 is easily emitted from the sound hole 131b. As a result, the difference in the propagation distance between the acoustic signal AC1 and the acoustic signal AC2 mentioned above can be easily adjusted by the size, the number, the arrangement, and the like of the sound holes 131b. Note that the sound hole 131b is, for example, a sound hole penetrating the reflector 13, but the disclosed technology is not limited thereto. The sound hole 131b may not be through-hole as long as the acoustic signals inside the reflector 13 can be led to the outside. Here, in order to simplify the description, a case will be illustrated in which the shape of the edge of the open end of the sound hole 131b is a quadrangle (open end is rectangular), but the disclosed technology is not limited thereto. For example, the shape of the edge of the open end of the sound hole 131b may be another shape such as a circle, an ellipse, or a triangle. In addition, the open end of the sound hole 131b may be mesh-like.

With the above configuration, the acoustic signal AC1 (first acoustic signal) is emitted from the D1 direction side (first side) of the driver unit 11 (first driver unit), and the acoustic signal AC2 (second acoustic signal) is emitted from the D2 direction side (the other side) of the driver unit 11 (first driver unit), so that an attenuation rate η₁₁ of the acoustic signal AC1 (first acoustic signal) at a position P2 (second point) with the position P1 (first point) as a reference can be made equal to or less than a predetermined value ηₜₕ, or an attenuation amount η₁₂ of the acoustic signal AC1 (first acoustic signal) at the position P2 (second point) with the position P1 (first point) as a reference can be made equal to or more than a predetermined value ωₜₕ. Here, the position P1 (first point) is a predetermined point at which the acoustic signal AC1 (first acoustic signal) reaches. On the other hand, the position P2 (second point) is a predetermined point where a distance from the acoustic signal output apparatus 10 is farther than the position P1 (first point). The predetermined value ηₜₕ is a value smaller (lower value) than the attenuation rate η₂₁ of an arbitrary or specific acoustic signal (sound) due to air propagation at the position P2 (second point) with reference to the position P1 (first point). In addition, the predetermined value ωₜₕ is a value larger than the attenuation amount η₂₂ of an arbitrary or specific acoustic signal (sound) due to air propagation at the position P2 (second point) with reference to the position P1 (first point). That is, the acoustic signal output apparatus 10 is designed so that the attenuation rate η₁₁ is equal to or less than the predetermined value ηₜₕ smaller than the attenuation rate η₂₁, or the attenuation amount η₁₂ is equal to or more than the predetermined value ωₜₕ larger than the attenuation amount η₂₂. Note that the acoustic signal AC1 propagates through the air from the position P1 to the position P2, and attenuates due to this air propagation and the acoustic signal AC2. The attenuation rate η₁₁ is a ratio (AMP₂(AC1)/AMP₁(AC1)) of the magnitude AMP₂ (AC1) of the acoustic signal AC1 at the position P2, which attenuates due to air propagation and the acoustic signal AC2, with respect to the magnitude AMP₁ (AC1) of the acoustic signal AC1 at the position P1. In addition, the attenuation amount η₁₂ is the difference (|AMP₁ (AC1)−AMP₂ (AC1)|) between the magnitude AMP₁ (AC1) and the magnitude AMP₂ (AC1). On the other hand, when the acoustic signal AC2 is not assumed, an arbitrary or specific acoustic signal ACₐᵣ that propagates through the air from the position P1 to the position P2 attenuates due to the air propagation, not due to the acoustic signal AC2. The attenuation rate η₂₁ is the ratio (AMP₂ (ACₐᵣ)/AMP₁ (ACₐᵣ)) of the magnitude AMP₂ (ACₐᵣ) of the acoustic signal ACₐᵣ at the position P2, which attenuates due to air propagation (attenuates not due to the acoustic signal AC2) with respect to the magnitude AMP₁ (ACₐᵣ) of the acoustic signal ACₐᵣ at the position P1. In addition, the attenuation amount η₂₂ is the difference (|AMP₁ (ACₐᵣ)−AMP₂ (ACₐᵣ)|) between the magnitude AMP₁ (ACₐᵣ) and the magnitude AMP₂ (ACₐᵣ). Note that an example of the magnitude of the acoustic signal is the sound pressure of the acoustic signal or the energy of the acoustic signal. In addition, “sound leakage component” means, for example, a component having a high possibility of arriving at a region (for example, a person other than the user existing in the D1 direction) other than the user existing in the D1 direction among the acoustic signals AC1 emitted from the sound hole 161a. For example, “sound leakage component” may be a component propagating to a region other than a specific region in the D1 direction side or a component propagating to a region other than the D1 direction side among the acoustic signals AC1.

In addition, as illustrated in FIG. 1, FIG. 4, and the like, a sound hole 131aa (reflector sound hole) connected to the internal space of the housing 16 is provided on the bottom part 131a side (D2 direction side) of the reflector 13. The sound hole 131aa is, for example, a sound hole penetrating the reflector 13, but the disclosed technology is not limited thereto. The sound hole 131aa may not be a through hole as long as the acoustic signal in the internal space of the housing 16 can be led to the inside of the reflector 13. The sound hole 131aa will be described later in detail.

Although the material constituting the reflector 13 is not limited, it is desirable that at least the inner wall surface 131 is made of a material reflecting the acoustic signal. For example, the reflector 13 may be made of a rigid body such as synthetic resin or metal, or may be made of an elastic body such as rubber.

### <Housing 16>

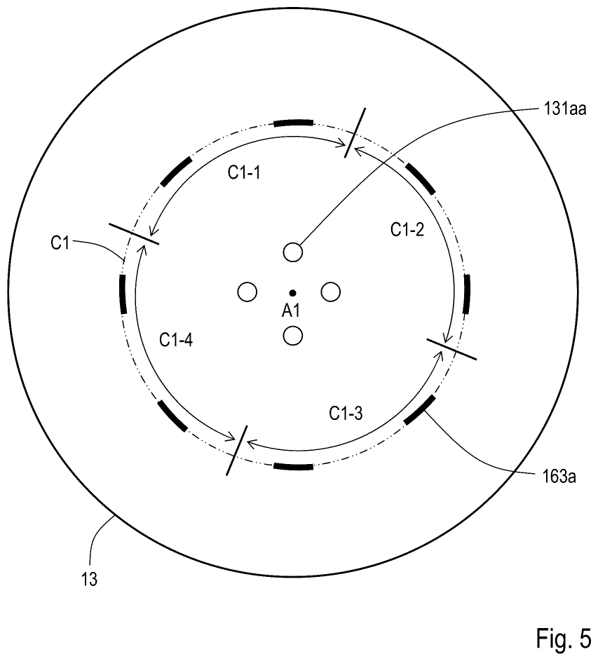

The housing 16 (second housing) is a hollow member having a wall part on the outside, and is placed outside the reflector 13. The housing 16 of the present embodiment is placed on the D2 direction side of the reflector 13. The driver unit 15 (second driver unit) is stored in the housing 16. The driver unit 15 of this example is fixed at a position separated by a fixed distance from a wall part 161 on the D1 direction side of the housing 16. By this, a hollow region AR0 is provided between a region AR1 inside the wall part 161 of the housing 16 and a surface 151 on the D1 direction side of the driver unit 15. The wall part of the housing 16 is provided with a sound hole 161a (third sound hole) for leading the acoustic signal AC3 (third acoustic signal) emitted from the driver unit 15 to the inside of the reflector 13 through the above-mentioned sound hole 131aa, and a single or a plurality of sound holes 163a (fourth sound hole) for leading the acoustic signal AC4 (fourth acoustic signal) emitted from the driver unit 15 to the outside of the reflector 13 among the outsides of the housing 16. In an example of the present embodiment, a concave part 161b is provided outside the wall part 161 on first side (D1 direction side) of the housing 16, and the outside of the bottom part 131a of the reflector 13 is fixed to the concave part 161b. The sound hole 161a (third sound hole) is provided in the concave part 161b and connected to the sound hole 131aa (reflector sound hole) of the reflector 13 (FIG. 1 and FIG. 4). By this, the acoustic signal AC3 emitted from the driver unit 15 to the region AR0 is led to the inside of the reflector 13 by the sound hole 161a and the sound hole 131aa. The acoustic signal AC3 led into the inside of the reflector 13 is emitted from the open end 130 of the reflector 13 in the D1 direction side. Note that it is desirable that the center of the sound hole 131aa (reflector sound hole) connected to the sound hole 161a (third sound hole) or a plurality of sound holes 131aa (reflector sound holes) connected to single or a plurality of sound holes 161a (third sound holes) is arranged on the axis line A1 (axis) or in the vicinity of the axis line A1 (axis) (for example, FIG. 5). As a result, the sound pressure of the acoustic signal AC3 emitted from the open end 130 of the reflector 13 becomes axis-symmetric or approximately axis-symmetric with respect to the axis line A1. In addition, the sound hole 163a faces an external space on the rear surface 132 side of the reflector 13, and the acoustic signal AC4 emitted to the hollow region AR (internal space) of the housing 16 on the D2 direction side of the driver unit 15 is led to the outside of the reflector 13 by the sound hole 163a. As mentioned above, the acoustic signal AC4 is the inverse phase signal or the approximate signal of the inverse phase signal of the acoustic signal AC3. In addition, the acoustic signal AC3 is the in-phase signal or the approximate signal of the in-phase signal of the acoustic signal AC1, and the acoustic signal AC4 is the in-phase signal or the approximate signal of the in-phase signal of the acoustic signal AC2. Therefore, at least a part of the acoustic signal AC4 emitted from the sound hole 163a and at least a part of sound leakage components of the acoustic signals AC1 and AC3 emitted from the open end 130 of the reflector 13 cancel each other out. By this, sound leakage, especially sound leakage on the low band side (acoustic signal AC3) can be suppressed. Note that the sound hole 161a and the sound hole 163a are, for example, sound holes penetrating the wall part of the housing 16, but the disclosed technology is not limited thereto. The sound hole 161a and the sound hole 163a may not be through-holes as long as the acoustic signal AC3 can be led to the inside of the reflector 13 and the acoustic signal AC4 can be led to the outside of the reflector 13. Although there is no limitation on the shape of the housing 16, for example, it is desirable that the shape of the housing 16 is rotation-symmetrical (line-symmetrical) or approximately rotation-symmetrical with a center of axis line A1. By this, the sound hole 163a can be easily provided so as to reduce the variation of sound pressure in each direction of the acoustic signal AC4 emitted from the housing 16. As a result, it is easy to uniformly reduce sound leakage in each direction. For example, the housing 16 has the wall part 161 placed on first side (D1 direction side) of the driver unit 15, the wall part 162 placed on the other side (D2 direction side) of the driver unit 15, and the wall part 163 surrounding a space sandwiched between the wall part 161 and the wall part 162 with a center of axis line A1 passing through the wall part 161 and the wall part 162 (FIG. 1 and FIG. 4). Here, in order to simplify the description, an example will be shown in which the housing 16 has an approximately cylindrical shape with both end surfaces. However, these are examples and do not intend to restrict the disclosed technology. For example, the housing 16 may have an approximately dome shape with a wall part at the end, a hollow approximately cubic shape, or any other three-dimensional shape. In addition, there is no limitation on the material that makes the housing 16. The housing 16 may be made of a rigid body such as synthetic resin or metal, or may be made of an elastic body such as rubber.

The user located in a specific region in the D1 direction side can hear the acoustic signals AC1 and AC3 emitted from the open end 130 of the reflector 13. As mentioned above, the acoustic signal AC2 which is the inverse phase signal or the approximate signal of the inverse phase signal of the acoustic signal AC1 is emitted from the sound hole 131b. In addition, the acoustic signal AC4 which is the inverse phase signal or the approximate signal of the inverse phase signal of the acoustic signal AC3 is emitted from the sound hole 163a. Here, a part of the emitted acoustic signals AC2 and AC4 and a part of the acoustic signals AC1 and AC3 emitted from the open end 130 of the reflector 13 (leakage components) cancel each other out. For example, a part of the acoustic signal AC2 mainly cancels out a part of the acoustic signal AC1, and a part of the acoustic signal AC4 mainly cancels out a part of the acoustic signal AC3. That is, the acoustic signal AC1 (first acoustic signal) is emitted from the D1 direction side (first side) of the driver unit 11 (first driver unit), the acoustic signal AC2 (second acoustic signal) is emitted from the D2 direction side (the other side) of the driver unit 11 (first driver unit), the acoustic signal AC3 (third acoustic signal) is emitted from the D1 direction side (first side) of the driver unit 15 (second driver unit), and the fourth acoustic signal is emitted from the D2 direction side (the other side) of the driver unit 15 (second driver unit), so that the attenuation rate η₁₁₂ of the acoustic signal AC1 (first acoustic signal) and the acoustic signal AC3 (third acoustic signal) at the position P2 (second position) with reference to the position P1 (first position) can be set to a predetermined value ηₜₕ or less, or the attenuation amount η₁₂₂ of the acoustic signal AC1 (first acoustic signal) and the acoustic signal AC3 (third acoustic signal) at the position P2 (second position) with reference to the position P1 (first position) can be set to a predetermined value ωₜₕ or more. Here, the position P1 (first point) is a predetermined point where the emitted acoustic signal AC1 (first acoustic signal) and acoustic signal AC3 (third acoustic signal) reach. On the other hand, the position P2 (second point) is a predetermined point where a distance from the acoustic signal output apparatus 10 is farther than that from the position P1 (first point). The predetermined value ηₜₕ is a value smaller (lower value) than the attenuation rate η₂₁ of an arbitrary or specific acoustic signal (sound) due to air propagation at the position P2 (second point) with reference to the position P1 (first point). In addition, the predetermined value ωₜₕ is a value larger than the attenuation amount η₂₂ of an arbitrary or specific acoustic signal (sound) due to air propagation at the position P2 (second point) with reference to the position P1 (first point). That is, the acoustic signal output apparatus 10 of the present embodiment is designed so that the attenuation rate η₁₁₂ is equal to or less than the predetermined value ηₜₕ smaller than the attenuation rate η₂₁, or the attenuation amount η₁₂₂ is equal to or more than the predetermined value ωₜₕ larger than the attenuation amount η₂₂. Note that the acoustic signal AC1 and the acoustic signal AC3 propagate through the air from the position P1 to the position P2, and attenuates due to this air propagation, the acoustic signal AC2, and the acoustic signal AC4. The attenuation rate η₁₁₂ is the ratio (AMP₂ (AC1)/AMP₁ (AC1)|) of the magnitude AMP₂ (AC1) of the acoustic signal AC1 at the position P2 attenuated due to the air propagation, the acoustic signal AC2, and the acoustic signal AC4 with respect to the magnitude AMP₁ (AC1) of the acoustic signal AC1 at the position P1, or the ratio (AMP₂ (AC3)/AMP₁ (AC13)|) of the magnitude AMP₂ (AC3) of the acoustic signal AC3 at the position P2 attenuated due to the air propagation, the acoustic signal AC2, and the acoustic signal AC4 with respect to the magnitude AMP₁ (AC3) of the acoustic signal AC3 at the position P1. Alternatively, the attenuation rate η₁₁₂ may be a statistical value (average value, addition value, multiplication value, and the like) of the ratio (AMP₂ (AC1)/AMP₁ (AC1)|) and the ratio (AMP₂ (AC3)/AMP₁ (AC13)). In addition, the attenuation amount η₁₂₂ is the difference (|AMPᵢ(AC1)−AMP₂ (AC1)|) between the magnitude AMP₁ (AC1) and the magnitude AMP₂ (AC1), or the difference (|AMP₁ (AC3)−AMP₂ (AC3)|) between the magnitude AMP₁ (AC3) and the magnitude AMP₂ (AC3). Alternatively, the attenuation η₁₂₂ may be a statistical value (average value, addition value, multiplication value, and the like) of the difference (|AMP₁ (AC1)−AMP₂ (AC1)|) and the difference (|AMP₁ (AC3)−AMP₂ (AC3)|). On the other hand, when the acoustic signal AC2 and the acoustic signal AC4 are not assumed, an arbitrary or specific acoustic signal ACₐᵣ that propagates through the air from the position P1 to the position P2 attenuates due to the air propagation, not due to the acoustic signal AC2 or the acoustic signal AC4. The attenuation rate η₂₁ is the ratio (AMP₂ (ACₐᵣ)/AMP₁ (ACₐᵣ)) of the magnitude AMP₂ (ACₐᵣ) of the acoustic signal ACₐᵣ at the position P2, which attenuates due to air propagation (attenuates not due to the acoustic signal AC2) with respect to the magnitude AMP₁ (ACₐᵣ) of the acoustic signal ACₐᵣ at the position P1. In addition, the attenuation amount η₂₂ is the difference (|AMP₁ (ACₐᵣ)−AMP₂ (ACₐᵣ)|) between the magnitude AMP₁ (ACₐᵣ) and the magnitude AMP₂ (ACₐᵣ).

By the configuration above, the sound leakage can be suppressed. In particular, the size of the driver unit 11 (first driver unit) is smaller than the size of the driver unit 15 (second driver unit). In addition, the driver unit 11 is placed inside the reflector 13, and the acoustic signals AC1 and AC2 emitted from the driver unit 11 are emitted from the open end 130 and the sound hole 131b of the reflector 13. On the other hand, the driver unit 15 is stored inside the housing 16 located outside the reflector 13, and the acoustic signal AC3 emitted from the driver unit 15 is led to the inside of the reflector 13, and further emitted from the open end 130 of the reflector 13. In contrast to this, the acoustic signal AC4 emitted from the driver unit 15 is emitted to the outside of the reflector 13 from the sound hole 163a of the housing 16. Therefore, the difference between the propagation distance until the acoustic signal AC1 emitted from the D1 direction side of the diaphragm 113 of the driver unit 11 reaches the position P2 (second point) and the propagation distance until the acoustic signal AC2 (second acoustic signal) emitted from the D2 direction side (the other side) of the diaphragm 113 reaches the position P2 (second point) is smaller than the difference between the propagation distance until the acoustic signal AC3 emitted from the D1 direction side (first side) of the diaphragm 153 of the driver unit 15 reaches the position P2 (second point) and the propagation distance until the acoustic signal AC4 emitted from the D2 direction side (the other side) of the diaphragm 153 reaches the position P2 (second point). Here, as the difference in propagation distance becomes smaller, the phase difference between the inverse phase wave (acoustic signal AC2 and acoustic signal AC4) and the reproduced sound (acoustic signal AC1 and acoustic signal AC3) at the position P2 becomes larger, and the sound leakage prevention effect is improved. Therefore, in terms of size and arrangement, the sound leakage prevention effect on the driver unit 11 side is higher than that of the driver unit 15 side. On the other hand, as the frequency is higher, the sound leakage prevention effect tends to be more affected by the difference in the propagation distance, so that as the frequency is higher, the sound leakage prevention effect tends to decrease more. Here, the driver unit 11 mainly takes charge of the high band acoustic signal among the reproduced acoustic signals, and the driver unit 15 mainly takes charge of the low band acoustic signal among the reproduced acoustic signals. Therefore, the sound leakage prevention effect on the driver unit 15 side is higher than that on the driver unit 11 side in terms of the frequency. By the characteristics of these sound leakage prevention effects, sufficient sound leakage prevention effects can be obtained in a wide frequency band. In addition, since the diameter of the diaphragm 153 (second diaphragm) of the driver unit 15 is larger than the diameter of the diaphragm (first diaphragm) of the driver unit 11, the sound pressure of a bass on the driver unit 15 side can be made larger than that of the driver unit 11. By these, the sound pressure in the low band can be sufficiently obtained while suppressing sound leakage.

<Arrangement of Sound Holes 161a and 163a>

The arrangement of the sound holes 161a and 163a will be illustrated.

The sound hole 161a (third sound hole) illustrated here is provided in the region AR1 (first region) of the wall part 161 placed on first side (D1 direction side which is a side from which the acoustic signal AC3 is emitted) of the driver unit 15 (FIG. 1 and FIG. 4). That is, the sound hole 161a opens toward the D1 direction (first direction) along the axis line A1, and connects to the sound hole 131aa of the reflector 13. In addition, the sound hole 163a (fourth sound hole) illustrated here is provided in the region AR3 of the wall part 163 contacting with the region AR between the region AR1 (first region) of the wall part 161 of the housing 16 and the region AR2 (second region) of the wall part 162 placed on the D2 direction side (the other side which is a side from which the acoustic signal AC4 is emitted) of the driver unit 15. That is, when a direction between the D1 direction (first direction) and the direction inverse to the D1 direction is defined as a D12 direction (second direction) with reference to the center of the housing 16 (FIG. 4), the sound hole 161a (third sound hole) is provided in the D1 direction side (first direction side) of the housing 16, and the sound hole 163a (fourth sound hole) is provided on the D12 direction side (second direction side) of the housing 16. For example, the housing 16 has the wall part 161 placed on first side (D1 direction side) of the driver unit 15, the wall part 162 placed on the other side (D2 direction side) of the driver unit 15, and the wall part 163 (side surface) surrounding the space sandwiched between the wall part 161 and the wall part 162 with the axis line A1 passing through the wall part 161 and the wall part 162 along the emission direction (D1 direction) of the acoustic signal AC3 as a center (FIG. 4), the sound hole 161a (third sound hole) is provided on the wall part 161, and the sound hole 163a (fourth sound hole) is provided on the wall part 163 (side surface). In addition, in this example, it is desirable that no sound hole is provided on the wall part 162 side of the housing 16. The reason is that the sound pressure level of the acoustic signal AC4 emitted from the housing 16 will exceed the level required to cancel out the sound leakage components of the acoustic signal AC3 when the sound hole is provided on the wall part 162 side of the housing 16, and the excess amount is perceived as sound leakage.

As illustrated in FIG. 1 or the like, the sound hole 161a illustrated here is arranged on the axis line A1 along the emission direction (D1 direction) of the acoustic signal AC3 or in the vicinity of that. The axis line A1 of this example passes through the center or the vicinity of the center of the region AR1 (first region) of the wall part 161 placed on first side (D1 direction side) of the driver unit 15 of the housing 16. For example, the axis line A1 is an axis line extending in the D1 direction through a central region of the housing 16. That is, the sound hole 161a of this example is provided at the center position of the region AR1 of the wall part 161 of the housing 16. In this example, in order to simplify the description, an example will be shown, in which the shape of the edge of the open end of the sound hole 161a is circular (open end is circular). However, this does not limit the disclosed technology. For example, the shape of the edge of the open end of the sound hole 161a may be another shape such as an ellipse, a quadrangle, or a triangle. In addition, the open end of the sound hole 161a may be mesh-like. In other words, the open end of the sound hole 161a may consist of a plurality of holes. In addition, in this example, in order to simplify the description, an example will be shown in which four sound holes 161a are provided in the region AR1 (first region) of the wall part 161 of the housing 16. However, this does not limit the disclosed technology. For example, one or more sound holes 161a may be provided in the region AR1 (first region) of the wall part 161 of the housing 16, and other numbers of sound holes 161a may be provided.

It is desirable that the sound hole 163a (fourth sound hole) is arranged in consideration of the following viewpoints, for example.
(1) Viewpoint of position: the sound hole 163a is arranged so that the propagation path of the acoustic signal AC4 emitted from the sound hole 163a overlap the propagation path of the sound leakage components of the acoustic signal AC3 to cancel it out.
(2) Viewpoint of area: the propagation region of the acoustic signal AC4 emitted from the sound hole 163a and the frequency characteristics of the housing 16 differ depending on the opening area of the sound hole 163a. In addition, the frequency characteristics of the housing 16 affect the frequency characteristics of the acoustic signal AC4 emitted from the sound hole 163a, that is, affect the amplitude at each frequency. Considering the propagation region and frequency characteristics of the acoustic signal AC4 emitted from this sound hole 163a, the opening area of the sound hole 163a is determined so that the sound leakage components are canceled out by the acoustic signal AC4 emitted from the sound hole 163a in the region where the sound leakage component is to be canceled out.

From the above viewpoint, for example, it is desirable that the sound hole 163a (fourth sound hole) is configured as follows.

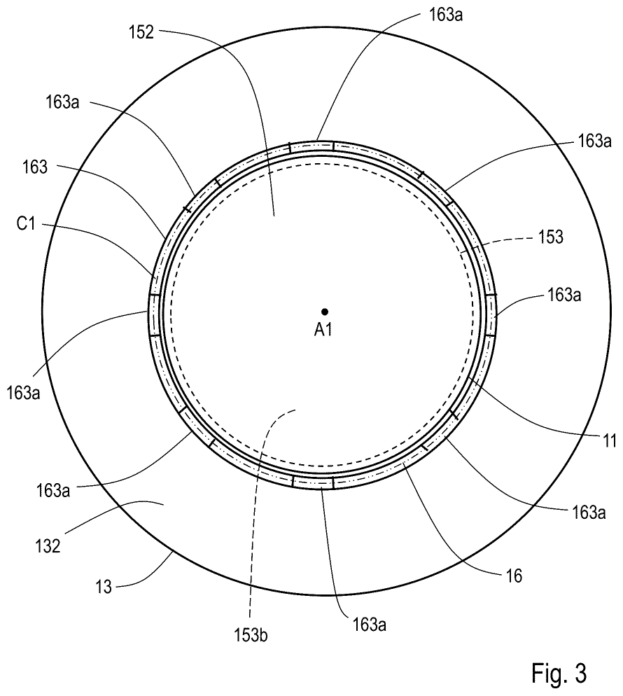

For example, as illustrated in FIG. 3 and FIG. 5, it is desirable that a plurality of sound holes 163a (fourth sound holes) is provided along the circumference (circle) C1 with the axis line A1 along the emission direction of the acoustic signal AC3 (first acoustic signal) as a center. When the plurality of sound holes 163a is provided along the circumference C1, the acoustic signal AC4 is radially (radially with the axis line A1 as a center) emitted from the sound holes 163a. Here, the sound leakage components of the acoustic signal AC3 are also radially (radially with the axis line A1 as a center) emitted from the sound hole 161a to the outside. Therefore, the plurality of sound holes 163a is provided along the circumference C1, so that the sound leakage components of the acoustic signal AC3 can be appropriately canceled out by the acoustic signal AC4. Here, in order to simplify the description, an example will be shown in which the plurality of sound holes 163a is provided on the circumference C1. However, it is sufficient that the plurality of sound holes 163a is provided along the circumference C1, and it is not necessary that all the sound holes 163a are arranged strictly on the circumference C1.

In addition, preferably, when the circumference C1 is equally divided into a plurality of unit arc regions, a total opening area of the sound holes 163a (fourth sound holes) provided along a first arc region which is any of unit arc regions is the same as or approximately the same as a total opening area of the sound holes 163a (fourth sound holes) provided along a second arc region which is any of the unit arc regions excluding the first arc region. For example, as illustrated in FIG. 5, when the circumference C1 is equally divided into four unit arc regions C1-1, . . . , C1-4, a total opening area of the sound holes 163a (fourth sound holes) provided along a first arc region which is any of the unit arc regions C1-1, . . . , C1-4 (suppose C1-1) is the same as or approximately the same as a total opening area of the sound holes 163a (fourth sound holes) provided along a second arc region which is any of the unit arc regions excluding the first arc region (C1-2, for example). Note that, in order to simplify the description here, an example will be shown, in which the circumference C1 is equally divided into four unit arc regions C1-1, . . . , C1-4, but this does not limit the disclosed technology. In addition, “α1 and α2 are approximately the same” means that the difference between α1 and α2 is R % or less of al. Examples of R % are 3%, 5%, 10%, and the like. By this, the sound pressure distribution of the acoustic signal AC4 emitted from the sound hole 163a provided along the first arc region and the sound pressure distribution of the acoustic signal AC4 emitted from the sound hole 163a provided along the second arc region become axis-symmetric or approximately axis-symmetric with respect to the axis line A1. Preferably, the total opening areas for each unit arc region of the sound holes 163a (fourth sound holes) provided along each unit arc region are the same or approximately the same. As a result, the sound pressure distribution of the acoustic signal AC4 emitted from the sound holes 163a becomes axis-symmetric or approximately axis-symmetric with respect to the axis line A1. By this, the sound leakage components of the acoustic signal AC3 can be more appropriately canceled out by the acoustic signal AC4.

More preferably, it is desirable that the plurality of sound holes 163a is provided along the circumference C1 with the same shape, the same size, and the same interval. When the plurality of sound holes 163a is provided along the circumference C1 with the same shape, the same size, and the same interval, the sound leakage components of the acoustic signal AC3 can be more appropriately canceled out by the acoustic signal AC4. However, the disclosed technology is not limited thereto.

Here, in order to simplify the description, a case is illustrated, in which the shape of the edge of the open end of the sound hole 163a is a quadrangle (open end is rectangular), but the disclosed technology is not limited thereto. For example, the shape of the edge of the open end of the sound hole 163a may be another shape such as a circle, an ellipse, or a triangle. In addition, the open end of the sound hole 163a may be mesh-like. In other words, the open end of the sound hole 163a may consist of a plurality of holes. In addition, there is no limitation on the number of sound holes 163a, and a single sound hole 163a may be provided in the region AR3 of the wall part 163 of the housing 16, or a plurality of sound holes 163a may be provided.

<Cutoff Frequency of Reflector 13 with Driver Unit 11>

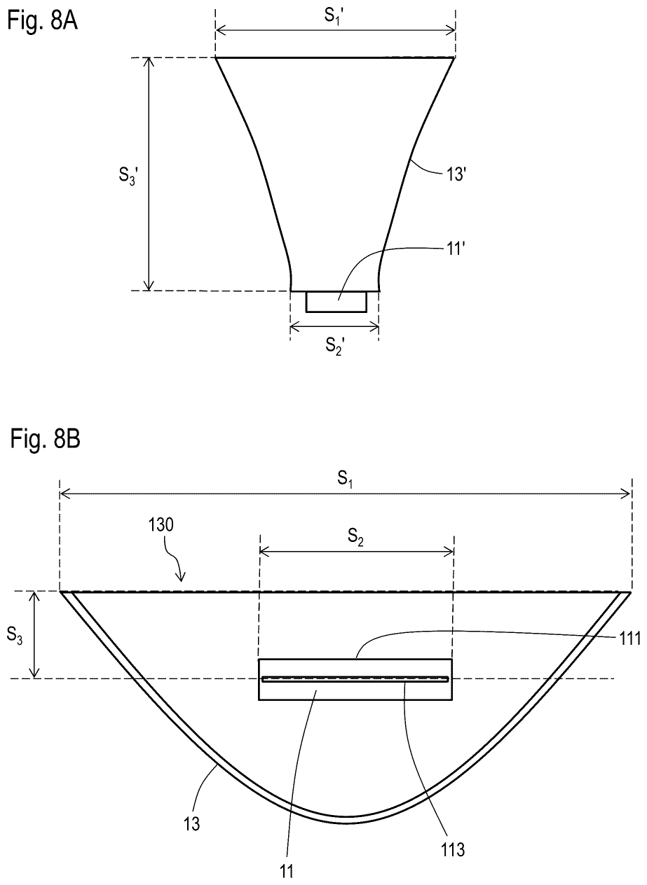

A cutoff frequency of the reflector 13 in which the driver unit 11 is placed will be considered. FIG. 8A illustrates a horn speaker in which a horn 13′ is attached to a driver unit 11′. Here, the opening area of the mouth (mouth) portion of the horn 13′ is defined as S₁′, the opening area of the throat (throat) portion of the horn 13′ is defined as S₂′, and the length of the horn 13′ is defined as S₃′. The driver unit 11′ is attached to the mouth portion of the horn 13′. The cutoff frequency f_{c} of this horn speaker is expressed by a following formula (1).

$$\begin{matrix}[ Math . 1 ] & \\ {f}_{c} = \frac{m c}{4 π} & ( 1 )\end{matrix}$$

Here, m represents a spread coefficient, and c represents a sound velocity. Note that the sound pressure of the acoustic signal emitted from the mouth portion of the horn speaker is rapidly lowered from the point exceeding the cutoff frequency f_{c}. That is, the cutoff frequency f_{c} represents the frequency characteristics of the acoustic signal that can be outputted from the horn speaker. Here, the following relation descried by formula (2) is known.

$$\begin{matrix}[ Math . 2 ] & \\ {S}_{2}^{′} = {S}_{1}^{′} {e}^{m {S}_{{3}^{′}}} & ( 2 )\end{matrix}$$

By rewriting the formula (2), the following formula (3) is obtained.

$$\begin{matrix}[ Math . 3 ] & \\ {mS}_{3}^{′} = {log}_{e} \frac{{S}_{2}^{′}}{{S}_{1}^{′}} & ( 3 )\end{matrix}$$

Further, by modifying the formula (3), the spread coefficient m can be approximated as shown in the following formula (4).

$$\begin{matrix}[ Math . 4 ] & \\ m \approx \frac{2.3}{{S}_{3}^{′}} {log}_{10} \frac{{S}_{2}^{′}}{{S}_{1}^{′}} & ( 4 )\end{matrix}$$

Although the reflector 13 of the present embodiment is different from the horn, it is considered that the cutoff frequency of the reflector 13 in which the driver unit 11 is placed exhibits a characteristic close to this. FIG. 8B illustrates the reflector 13 in which the driver unit 11 of the present embodiment is placed. Here, the opening area S₁ of the open end 130 of the reflector 13 is regarded as the opening area S₁′ of the mouth portion of the horn, the area S₂ of the surface 111 of the driver unit 11 is regarded as the opening area S₂′ of the throat portion of the horn, and the length S₃ from the surface 111 of the driver unit 11 to the open end 130 of the reflector 13 is regarded as the length S3′ of the horn. Then, the cutoff frequency f_{c} of the reflector 13 in which the driver unit 11 is placed can be approximated as shown by a following formula (5) from the formula (1) and the formula (4).

$$\begin{matrix}[ Math . 5 ] & \\ {f}_{c} \approx \frac{2.3 c}{4 π {S}_{3}} {log}_{10} \frac{{S}_{2}}{{S}_{1}} & ( 5 )\end{matrix}$$

That is, the reflector 13 in which the driver unit 11 is placed can be regarded as a speaker having the cutoff frequency f_{c} represented by the formula (5).

### <Reproducing Device 100 and Signal Separating Device 101>

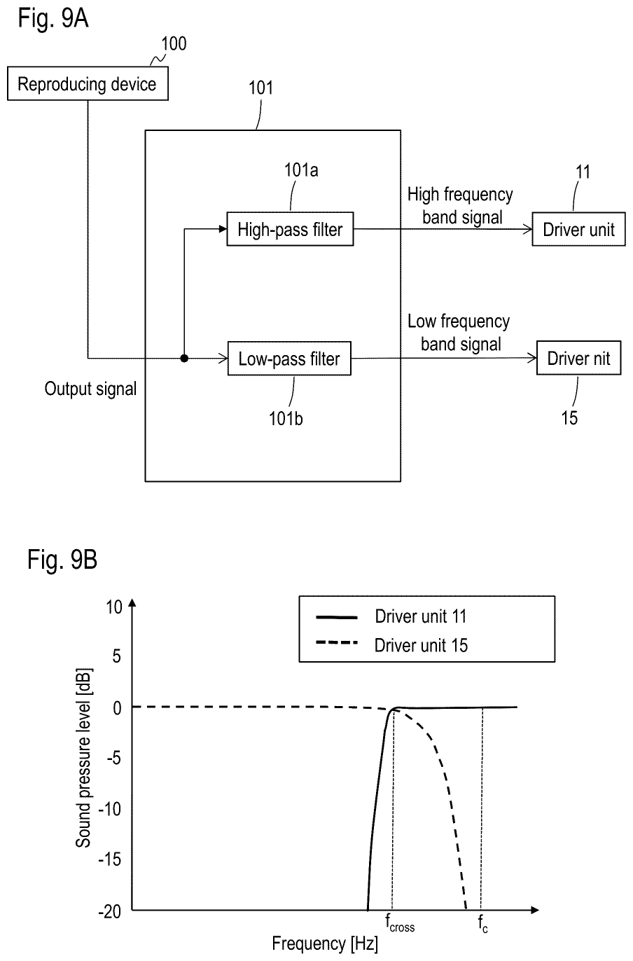

As illustrated in FIG. 9A, the output signal outputted from the reproducing device 100 is inputted to the signal separating device 101. The signal separating device 101 separates the inputted output signal into the high frequency band signal on the high band side and the low frequency band signal on the low band side. In the example in FIG. 9B, the output signal is branched into two, and the branched output signals are inputted to a high-pass filter 101a and a low-pass filter 101b, respectively. The high-pass filter 101a attenuates the low band side of the inputted output signal to obtain and output the high frequency band signal. The low-pass filter 101b attenuates the high band side of the inputted output signal to obtain and output the low frequency band signal. The high frequency band signal is inputted to the driver unit 11 of the acoustic signal output apparatus 10, and the driver unit 11 emits the acoustic signal AC1 to the D1 direction side and emits the acoustic signal AC2 to the D2 direction side. The low frequency band signal is inputted to the driver unit 15 of the acoustic signal output apparatus 10, and the driver unit 15 emits the acoustic signal AC3 to the D1 direction side and emits the acoustic signal AC4 to the D2 direction side.

As illustrated in FIG. 9B, in the present embodiment, the crossover frequency is defined as f_{cross}, the driver unit 11 is made to emit the acoustic signals AC1 and AC2 in the high frequency band having sufficient sound pressure at a frequency equal to or more than the crossover frequency f_{cross}, the driver unit 15 is made to emit the acoustic signals AC3 and AC4 of the low frequency band having sufficient sound pressure at a frequency equal to or less than the crossover frequency f_{cross}. That is, the low-pass filter 101b outputs a signal of the low frequency band having sufficient sound pressure at a frequency equal to or less than the crossover frequency f_{cross}. In addition, the high-pass filter 101a outputs a signal of the high frequency band having sufficient sound pressure at a frequency equal to or more than the crossover frequency f_{cross}. In this case, it is desirable that the crossover frequency f_{cross} is set to be lower than the cutoff frequency f_{c}, which is represented by the formula (5), of the speaker consisting of the driver unit 11 and the reflector 13. That is, it is desirable that the crossover frequency f_{cross} between the high frequency band and the low frequency band is lower than the cutoff frequency f_{c} represented by the formula (5). For example, one example of the crossover frequency f_{cross} is 1000 [Hz] or in its vicinity, and the cutoff frequency f_{c} is higher frequency than 1000 [Hz]. By this, sufficient sound pressure can be obtained in the high frequency band. Note that the crossover frequency f_{cross} and the cutoff frequency f_{c} may be determined so as to obtain an all-band signal having desired frequency characteristics at the hearing point of the user located in the D1 direction side.

### Experimental Results

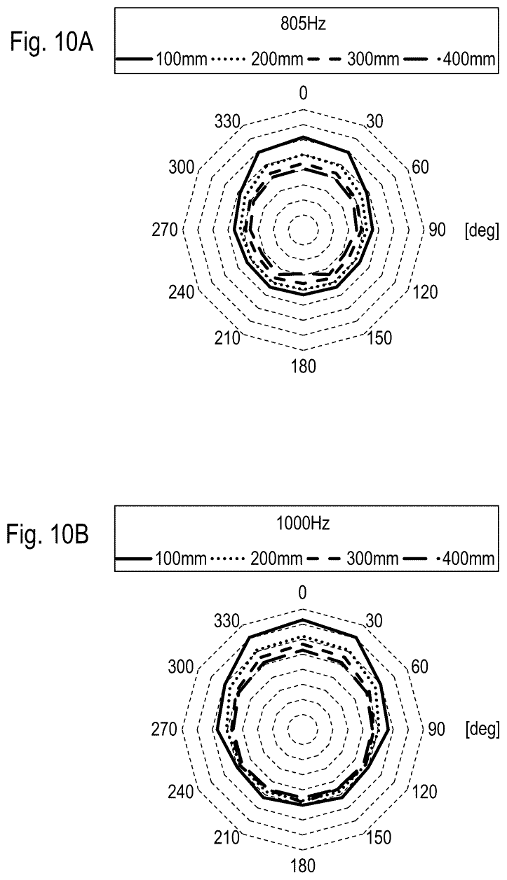

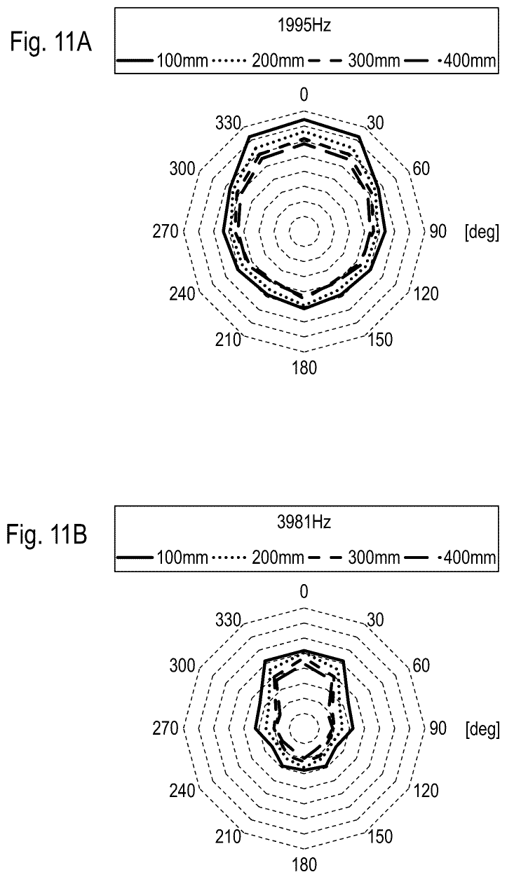

Experimental results will be shown. In FIG. 10A, FIG. 10B, FIG. 11A, FIG. 11B, and FIG. 12, graphs (radar charts) representing sound pressures at frequencies of 805 Hz, 1000 Hz, 1995 Hz, 3981 Hz, and 7943 Hz of the acoustic signal observed around the acoustic signal output apparatus 10 of the present embodiment are shown, respectively. 0 [deg] represents the D1 direction, 180 [deg] represents the D2 direction, and each line represents a sound pressure level at a position separated by 100 mm, 200 mm, 300 mm, and 400 mm in each direction from the acoustic signal output apparatus 10. In these graphs, the sound pressure level is lower as it is closer to the center, and the sound pressure level is higher as it is closer to the outside.

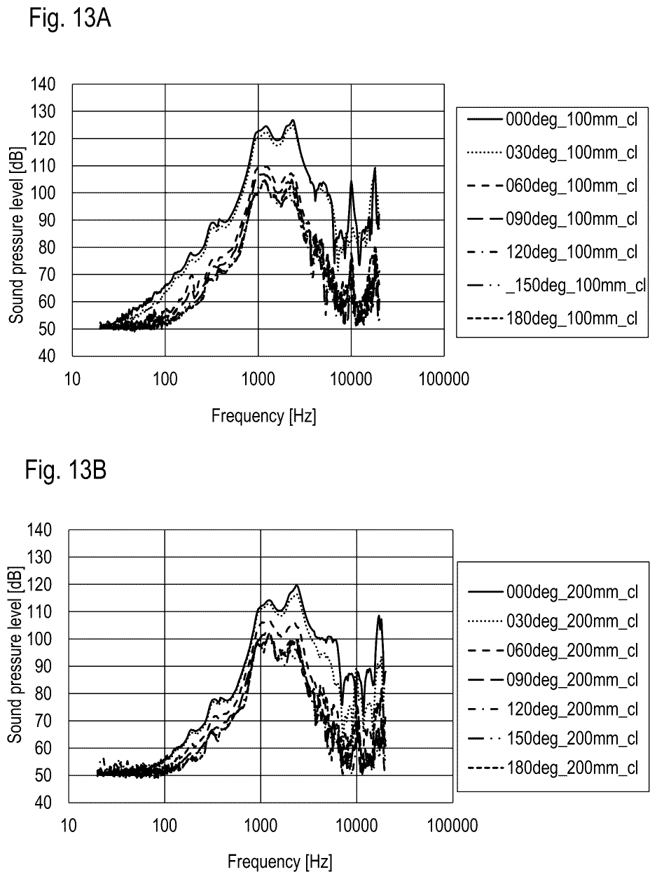

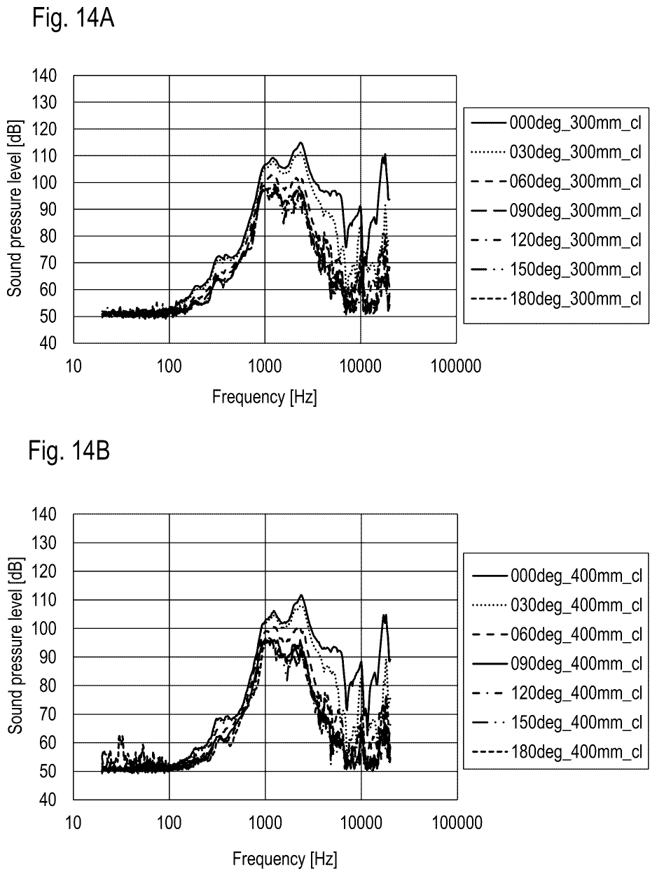

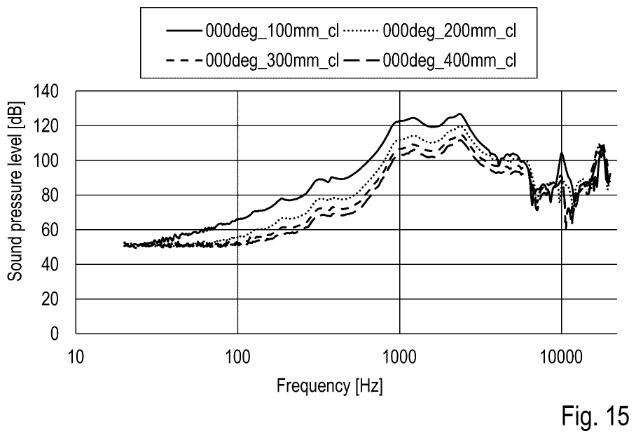

FIG. 13A to FIG. 15 show graphs representing the frequency characteristics of the acoustic signal observed around the acoustic signal output apparatus 10 of the present embodiment. In these graphs, the horizontal axis represents the frequency [Hz], and the vertical axis represents the sound pressure level [dB]. Each line represents the sound pressure level [dB] at each direction [deg] and each relative position [mm] with respect to the acoustic signal output apparatus 10. In the explanatory note of these graphs, “aaa deg_bbb mm cl” represents that a direction with respect to the acoustic signal output apparatus 10 is aaa [deg] and a sound pressure level [dB] observed at a position where a relative position is bbb [mm].

In this way, the acoustic signal output apparatus 10 of the present embodiment sufficiently suppresses the sound leakage to other positions while securing sufficient sound pressure in a specific region in the D1 direction side in a wide frequency band. Especially, the sound leakage to other positions can be sufficiently suppressed by the directivity of the reflector 13 while securing sufficient sound pressure in a specific region in the D1 direction side even in a high band exceeding 1,000 Hz. In this way, in the present embodiment, the sound leakage to the surroundings can be suppressed in the wide frequency band including the high band.

### [Modification Example 1 of First Embodiment of Localized Sound Speaker]

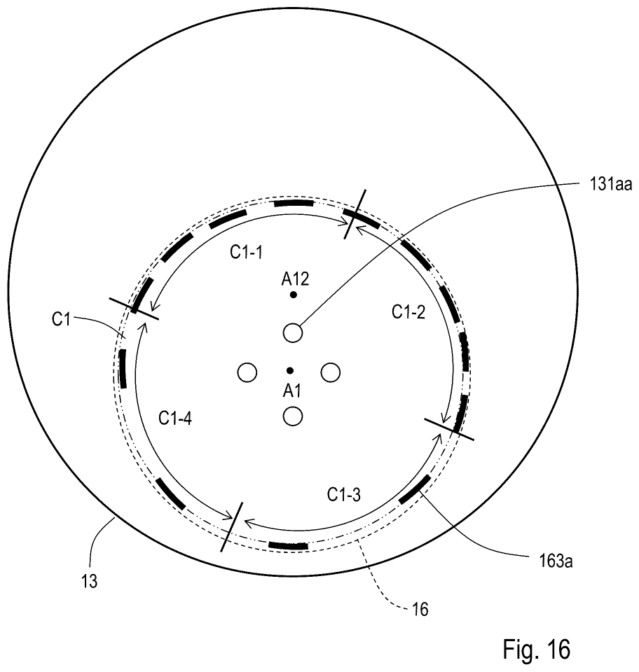

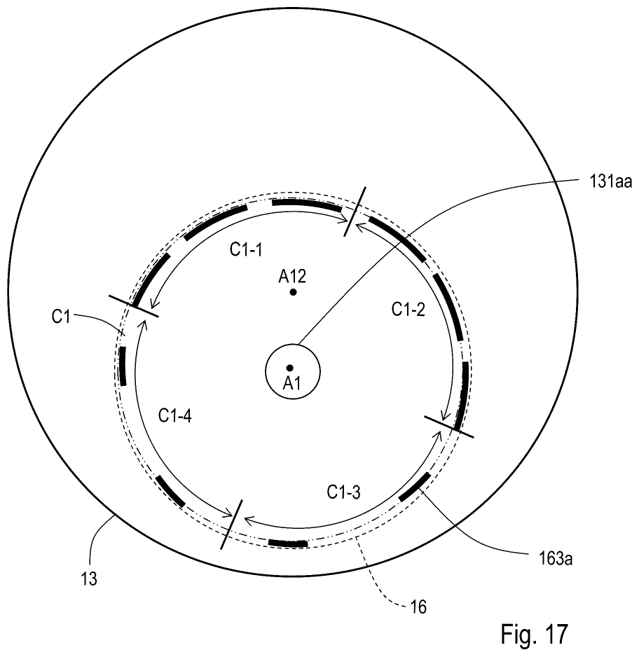

The matters different from above description are mainly described below, and the matters already described will be explained simply. As mentioned above, a single sound hole 161a may be provided in the region AR1 of the wall part 161 of the housing 16 or a plurality of sound holes 161a may be provided, or a single sound hole 131aa may be provided or a plurality of sound holes 131aa may be provided, which connects the sound hole 161a to the bottom part 131a of the reflector 13. In addition, the reflector 13 may be placed to an eccentric position (a position on an axis line A12 parallel to the axis line A1 deviated from the axis line A1) (hereinafter simply referred to as an “eccentric position”) deviated from the center (central position) of the housing 16. For example, as illustrated in FIG. 16, the center of the plurality of sound holes 161a and the sound holes 131aa may be placed on the axis line A1, and the reflector 13 may deviate on the axis line A12. Alternatively, as illustrated in FIG. 17, one sound hole 161a and sound hole 131aa may be arranged on the axis line A1, and the reflector 13 may deviate on the axis line A12. In other words, the reflector 13 may be placed eccentrically with respect to the housing 16 and one sound hole 161a and sound hole 131aa.

When the reflector 13 is placed eccentrically to the housing 16 and one sound hole 161a and sound hole 131aa, the distribution and opening area of the sound hole 163a may deviate in response to this. In the example in FIG. 16, the number of sound holes 163a provided along the unit arc regions C1-3 and C1-4 far from the axis line A12 is smaller than the number of sound holes 163a provided along the unit arc regions C1-1 and C1-2 closer to the axis line A12 than that. In the example in FIG. 17, each opening area of the sound hole 163a provided along the unit arc regions C1-3 and C1-4 far from the axis line A12 is smaller than each opening area of the sound hole 163a provided along the unit arc regions C1-1 and C1-2 closer to the axis line A12 than that. That is, when the circumference C1 is equally divided into a plurality of unit arc regions, the total opening area of the sound holes 163a (second sound holes) provided along the first arc region which is any of the unit arc regions (suppose C1-3 or C1-4) is smaller than the total opening area of the sound holes 163a provided along the second arc region which is any of the unit arc regions (C1-1 or C1-2 for example) closer to the axis line A12 than the first arc region. When the reflector 13 is eccentrically placed toward the eccentric position, the distribution of the acoustic signal AC3 emitted to the outside from the open end 130 of the reflector 13 also deviates toward the eccentric position. Here, by deviating the distribution and the opening area of the sound hole 163a toward the eccentric position, the distribution of the acoustic signal AC4 emitted to the outside from the sound holes 163a can also deviate toward the eccentric position. By this, the sound leakage components of the acoustic signal AC3 can be sufficiently canceled out by the emitted acoustic signal AC4.

### [Modification Example 2 of First Embodiment of Localized Sound Speaker]

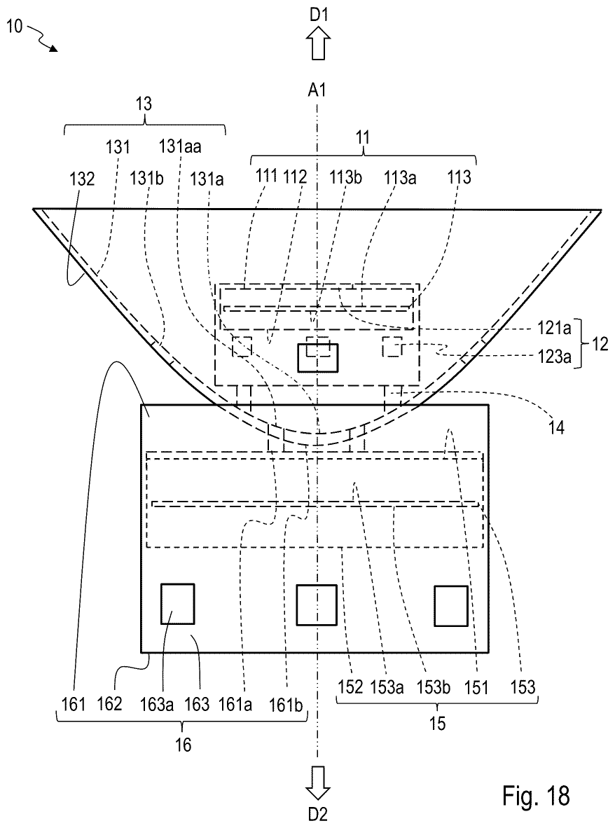

As illustrated in FIG. 18 to FIG. 21, the driver unit 11 (first driver unit) of the localized sound speaker in the first embodiment or the modification example 1 may be stored in a housing 12 (first housing) different from the housing 16 (second housing), and the housing 12 storing the driver unit 11 inside like this may be placed inside the reflector 13.

### <Housing 12>

The housing 12 is a hollow member having a wall part on the outside, sound holes 121a and 123a are provided on the wall part, and the driver unit 11 is stored inside. For example, the driver unit 11 is fixed to an end part in the D1 direction side inside the housing 12. Although there is no limitation on the shape of the housing 12, for example, it is desirable that the shape of the housing 12 is rotation-symmetrical (line-symmetrical) or approximately rotation-symmetrical with respect to the axis line A1. By this, the sound hole 123a can be easily provided so as to reduce the variation of energy of the acoustic signal emitted from the housing 12 in each direction. For example, the housing 12 has a first end surface that is a wall part 121 placed in first side (D1 direction side) of the driver unit 11, a second end surface that is a wall part 122 placed in the other side (D2 direction side) of the driver unit 11, and a side surface that is a wall part 123 that surrounds the space sandwiched between the first end surface and the second end surface with the axis line A1 passing through the first end surface and the second end surface as a center. Here, in order to simplify the description, an example will be shown in which the housing 12 has an approximately cylindrical shape with both end surfaces. However, these are examples and do not intend to restrict the disclosed technology. For example, the housing 12 may have an approximately dome shape with a wall part at the end, a hollow approximately cubic shape, or any other three-dimensional shape. In addition, there is no limitation on the material that makes the housing 12. The housing 12 may be made of a rigid body such as synthetic resin or metal, or may be made of an elastic body such as rubber.

<Sound Holes 121a and 123a>

As mentioned above, the wall part of the housing 12 is provided with a sound hole 121a (first sound hole) for leading the acoustic signal AC1 (first acoustic signal) emitted from the driver unit 11 to the outside (inside of the reflector 13) and a sound hole 123a (second sound hole) for leading the acoustic signal AC2 (second acoustic signal) emitted from the driver unit 11 to the outside (inside of the reflector 13). The sound hole 121a and the sound hole 123a are, for example, through-holes penetrating the wall part of the housing 12, but the disclosed technology is not limited thereto. The sound hole 121a and the sound hole 123a may not be through-holes, as long as the acoustic signal AC1 and the acoustic signal AC2 can be respectively led to the outside (inside of the reflector 13).

The arrangement of the sound holes 121a and 123a will be illustrated.

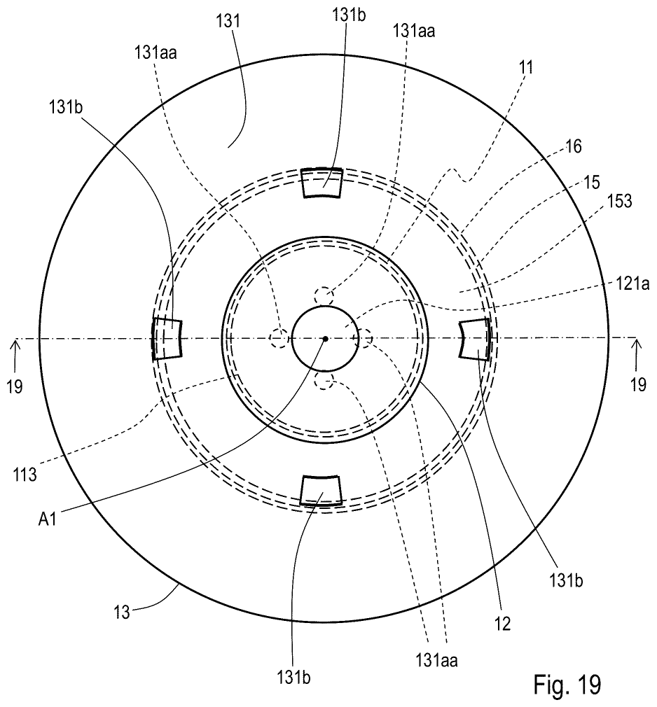

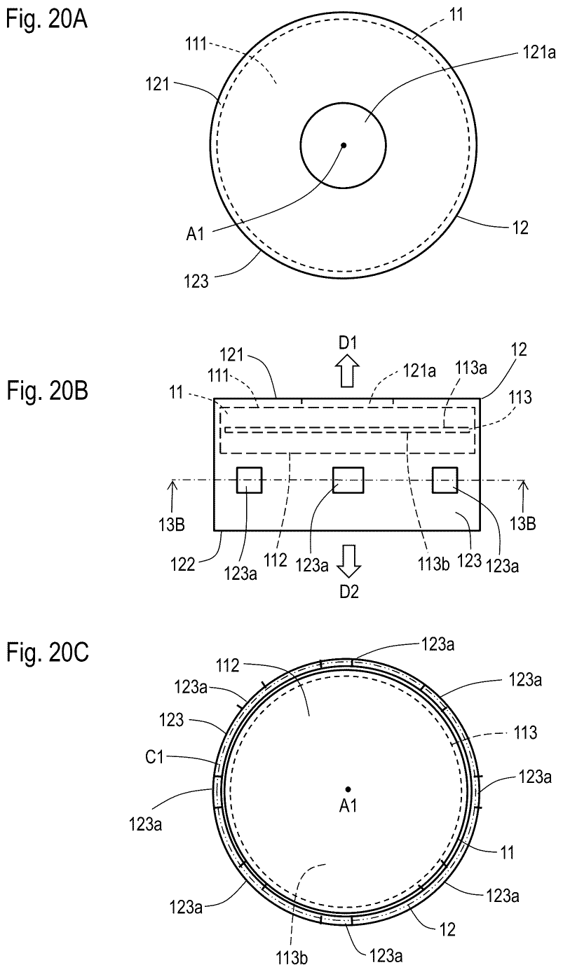

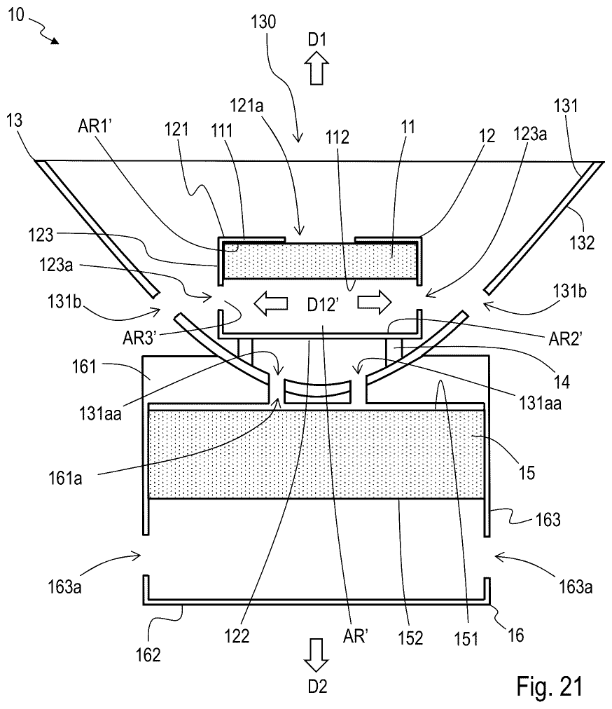

The sound hole 121a (first sound hole) illustrated here is provided in the region AR1 (first region) of the wall part 121 placed on first side (D1 direction side which is a side from which the acoustic signal AC1 is emitted) of the driver unit 11 (FIG. 18, FIG. 19, FIG. 20A, FIG. 20B, and FIG. 21). That is, the sound hole 121a opens toward the D1 direction (first direction) along the axis line A1. In addition, the sound hole 123a (second sound hole) illustrated here is provided in a region AR3′ of the wall part 123 contacting with a region AR′ between a region AR1′ of the wall part 121 of the housing 12 and a region AR2′ of the wall part 122 placed in the D2 direction side (the other side which is a side from which the acoustic signal AC2 is emitted) of the driver unit 11. That is, when, with reference to the center of the housing 12, a direction between the D1 direction (first direction) and a direction inverse to the D1 direction is defined as a D12′ direction (second direction) (FIG. 21B), the sound hole 121a (first sound hole) is provided in the D1 direction side (first direction side) of the housing 12, and the sound hole 123a (second sound hole) is provided in the D12′ direction side (second direction side) of the housing 12. For example, when the housing 12 has the wall part 121 placed in first side (D1 direction side) of the driver unit 11, the wall part 122 placed in the other side (D2 direction side) of the driver unit 11, and the wall part 123 (side surface) that surrounds the space sandwiched between the wall part 121 and the wall part 122 with the axis line A1 passing through the wall part 121 and the wall part 122 along the emission direction (D1 direction) of the acoustic signal AC1 as a center (FIG. 18), the sound hole 121a (first sound hole) is provided on the wall part 121, and the sound hole 123a (second sound hole) is provided on the wall part 123 (side surface).

As illustrated in FIG. 18A or the like, the sound hole 121a illustrated here is arranged on the axis line A1 along the emission direction (D1 direction) of the acoustic signal AC1 or in the vicinity of it. That is, the sound hole 121a of this example is provided at the center position of the region AR1 of the wall part 121 of the housing 12. In this example, in order to simplify the description, an example will be shown, in which the shape of the edge of the open end of the sound hole 121a is circular (open end is circular). However, this does not limit the disclosed technology. For example, the shape of the edge of the open end of the sound hole 121a may be another shape such as an ellipse, a quadrangle, or a triangle. In addition, the open end of the sound hole 121a may be mesh-like. In other words, the open end of the sound hole 121a may consist of a plurality of holes. In addition, in this example, in order to simplify the description, an example will be shown, in which one sound hole 121a is provided in the region AR1 (first region) of the wall part 121 of the housing 12. However, this does not limit the disclosed technology. For example, two or more sound holes 121a may be provided in the region AR1 (first region) of the wall part 121 of the housing 12.

It is desirable that a plurality of sound holes 123a (second sound holes) is provided along the circumference (circle) C1 with the axis line A1 along the emission direction of the acoustic signal AC1 (first acoustic signal) as a center. Here, in order to simplify the description, an example will be shown, in which the plurality of sound holes 123a is provided on the circumference C1. However, it is sufficient that the plurality of sound holes 123a is provided along the circumference C1, and it is not necessary that all the sound holes 123a are arranged strictly on the circumference C1.

In addition, preferably, when the circumference C1 is equally divided into a plurality of unit arc regions, the total opening area of the sound holes 123a (second sound holes) provided along the first arc region which is any of the unit arc regions is the same as or approximately the same as the total opening area of the sound holes 123a (second sound holes) provided along the second arc region which is any of the unit arc regions excluding the first arc region.

More preferably, the plurality of sound holes 123a is provided along the circumference C1 with the same shape, the same size, and the same interval. When the plurality of sound holes 123a is provided along the circumference C1 with the same shape, the same size, and the same interval, the sound leakage components of the acoustic signal AC1 can be more appropriately canceled out by the acoustic signal AC2. However, the disclosed technology is not limited thereto.

Here, in order to simplify the description, a case will be illustrated, in which the shape of the edge of the open end of the sound hole 123a is a quadrangle (open end is rectangular), but the disclosed technology is not limited thereto. For example, the shape of the edge of the open end of the sound hole 123a may be another shape such as a circle, an ellipse, or a triangle. In addition, the open end of the sound hole 123a may be mesh-like. In other words, the open end of the sound hole 123a may consist of a plurality of holes. In addition, there is no limitation on the number of sound holes 123a, and a single sound hole 123a may be provided, or a plurality of sound holes 123a may be provided in the region AR3 of the wall part 123 of the housing 12.

The housing 12 is fixed to an inner wall surface 131 of the reflector 13 through the support part 14. In the present embodiment, a sound hole 121a side of the housing 12 placed inside the reflector 13 is directed toward the open end 130 side (D1 direction side) of the reflector 13, and the wall part 122 on the other side is directed toward the bottom part 131a side (D2 direction side) of the reflector 13. Preferably, it is desirable that at least a part of the sound holes 123a of the housing 12 is placed at positions facing the sound holes 131b of the reflector 13.

### [Modification Example 3 of First Embodiment of Localized Sound Speaker]

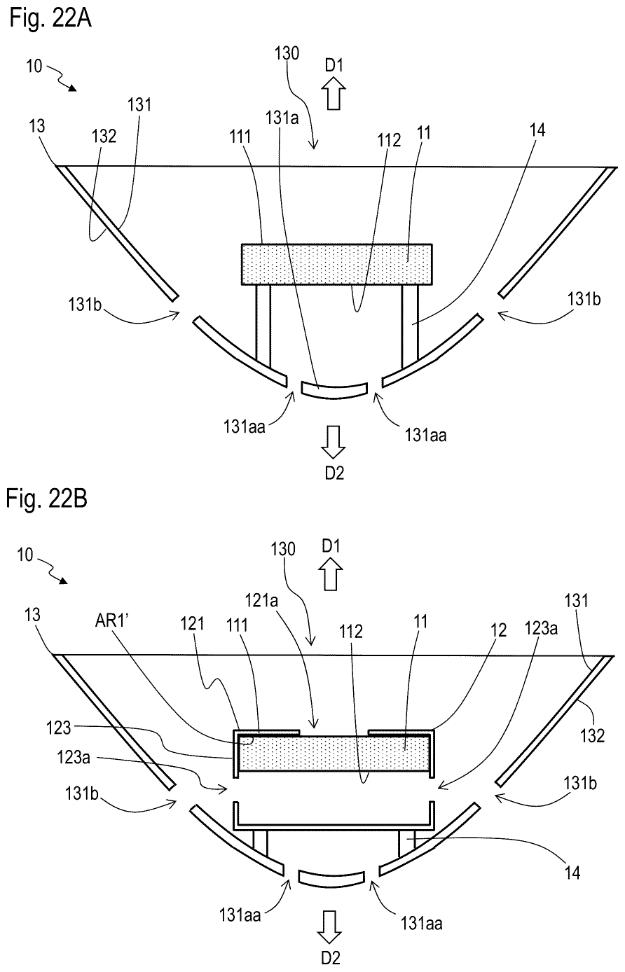

As illustrated in FIG. 22A and FIG. 22B, the housing 16 and the driver unit 15 (second driver unit) may be omitted in the first embodiment of the localized sound speaker and modification examples 1 and 2 thereof. In this case, the sound hole 131aa may be omitted.

### [Second Embodiment of Localized Sound Speaker]

Instead of the sound hole 131b or in addition to the sound hole 131b of the localized sound speaker in the first embodiment or the modification examples thereof, a notch part (slit part) 231b for opening the inside of the reflector 13 to the outside may be provided on a part of the open end 130 side of the reflector 13. As mentioned above, the acoustic signal AC1 and the acoustic signal AC2 are emitted from the open end 130 of the reflector 13. Here, the acoustic signal AC2 is the inverse phase signal or the approximate signal of the inverse phase signal of the acoustic signal AC1. Therefore, at a specific position P22 in the D1 direction side other than the position P21 where the user exists, a part of the acoustic signal AC1 cancels out a part of the acoustic signal AC2, and sound leakage of the acoustic signal AC1 at the position P22 is suppressed by this. However, in the high band components of the acoustic signal AC1 and the acoustic signal AC2, they hardly cancel each other out, and there is a case that, in the position P22, the acoustic signal AC2 conversely intensifies the acoustic signal AC1 and promotes sound leakage. To cope with this case, a notch part 231b is provided at a part of the open end 130 side of the reflector 13, so that sound leakage at the position P22 can be suppressed. The level of the sound pressure of the acoustic signal AC2 at the position P22 can be lowered by increasing the size of the notch part 231b. Therefore, the size of the notch part 231b may be designed so that the sound pressure of the acoustic signal AC2 (second acoustic signal) at the specific position P22 in the open end 130 direction of the reflector 13 becomes equal to or less than a predetermined level. For example, the size of the notch part 231b may be designed so that the sound pressure of the acoustic signal AC2 (second acoustic signal) in a frequency equal to or more than a predetermined frequency at the position P22 becomes a predetermined level or less. The notch part 231b will be illustrated below.

<Example 1 of Notch Part 231b (Notch Part 231b-SW)>

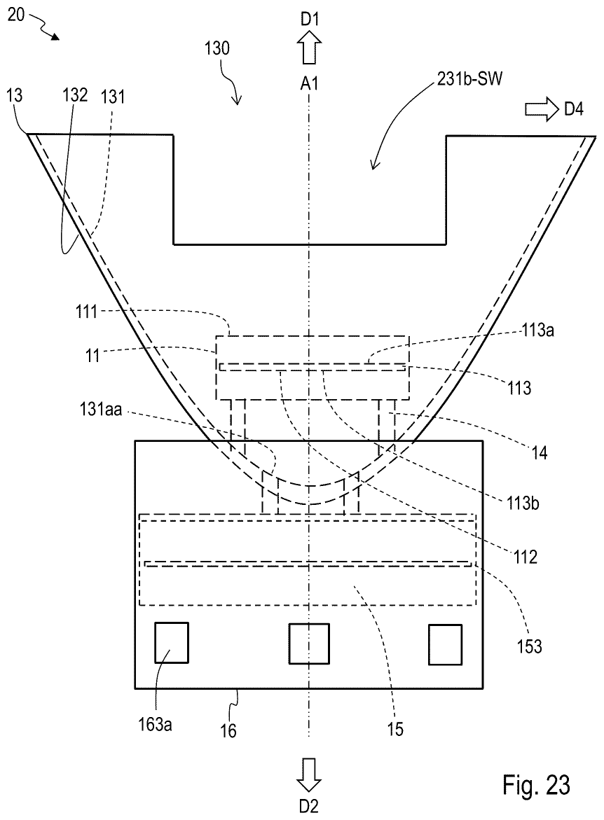

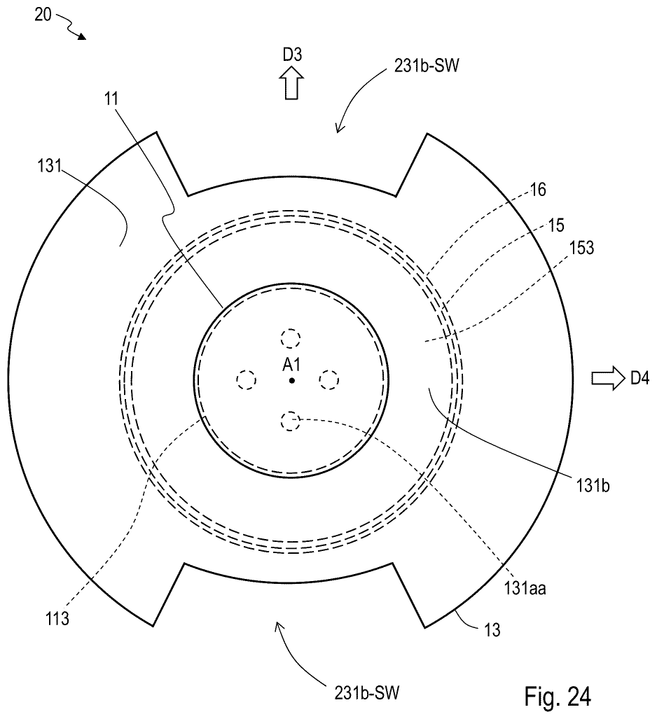

In the acoustic signal output apparatus 20 illustrated in FIG. 23 and FIG. 24, instead of the sound hole 131b, a laterally long notch part 231b-SW for opening the inside of the reflector 13 to the outside is provided in a part of the open end 130 side of the reflector 13. That is, the shape of the notch part 231b-SW of this example is longer in the D4 direction than in the D1-D2 direction orthogonal to D4 direction.

<Example 2 of Notch Part 231b (Notch Part 231b-LW)>

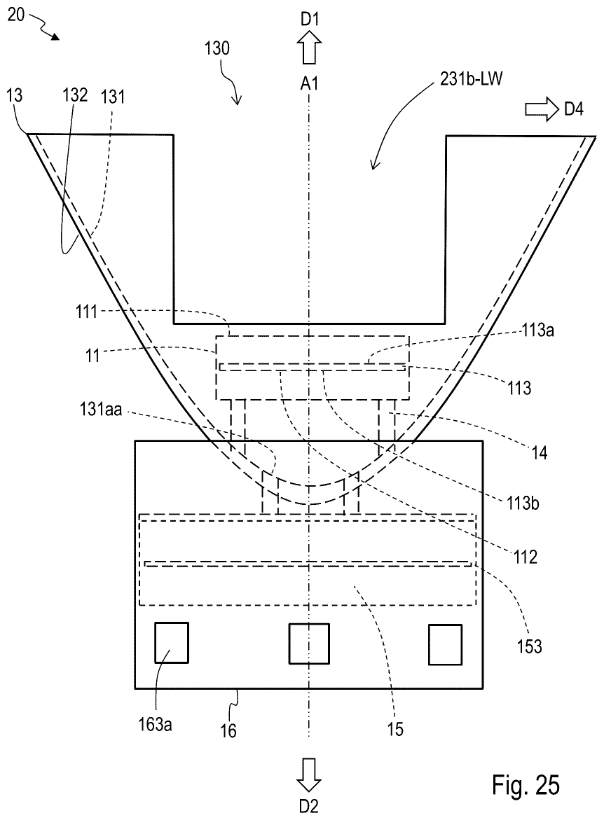

In the acoustic signal output apparatus 20 illustrated in FIG. 25, instead of the sound hole 131b, a longitudinally and laterally large notch 231b-LW opening the inside of the reflector 13 to the outside is provided in a part of the open end 130 side of the reflector 13. That is, the length of the notch part 231b-LW of this example in the D1-D2 direction is the same as the length of the notch part 231b-SW in FIG. 23 in the D1-D2 direction, but the length of the notch part 231b-LW in the D4 direction is longer than the length of the notch part 231b-SW in the D4 direction.

<Example 3 of Notch Part 231b (Notch Part 231b-LN)>

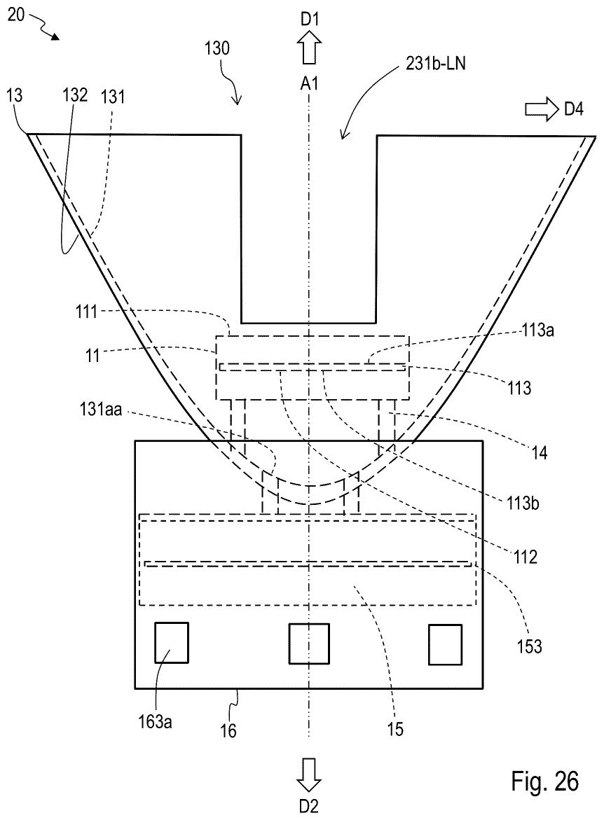

In the acoustic signal output apparatus 20 illustrated in FIG. 26, instead of the sound hole 131b, a longitudinally long notch part 231b-LN for opening the inside of the reflector 13 to the outside is provided at a part of the open end 130 side of the reflector 13. That is, the length of the shape of the notch part 231b-LN in the D1-D2 direction is the same as the length of the notch part 231b-LW in the D1-D2 direction in FIG. 25, but the length of the notch part 231b-LN in the D4 direction is shorter than the length of the notch part 231b-LW in the D4 direction.

### Experimental Results

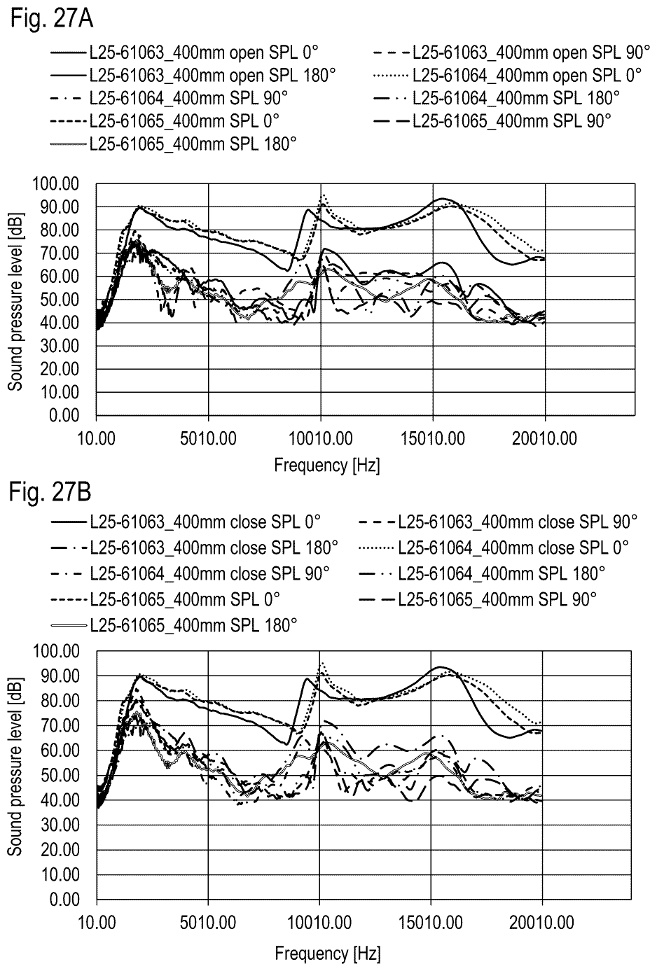

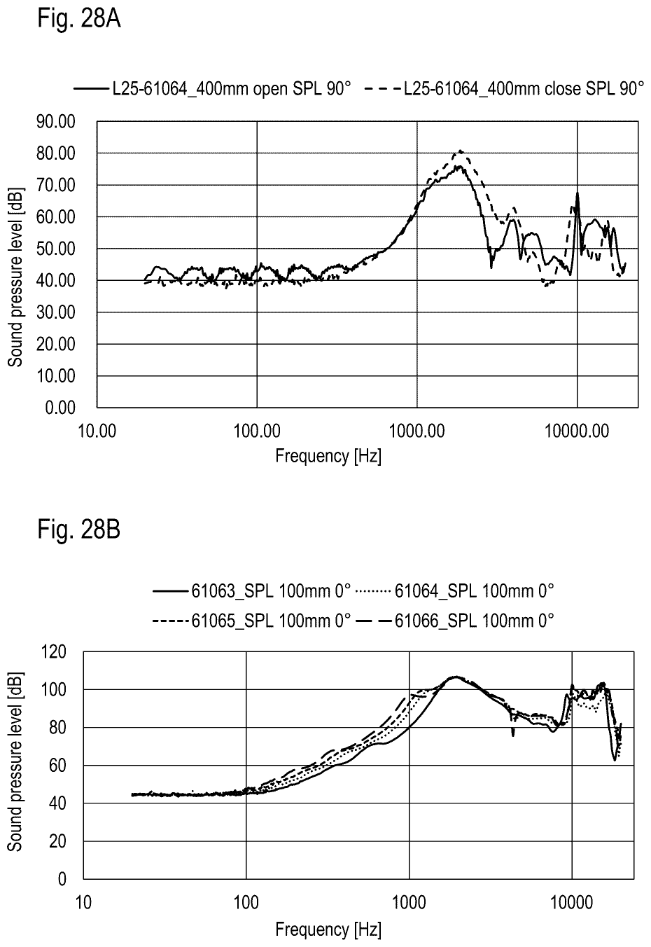

Experimental results will be shown in FIG. 27 and FIG. 28. The vertical axis represents the sound pressure level [dB], and the horizontal axis represents the frequency [Hz]. “L25-aaaaa_bbb mm. open SPL c°” of the explanatory note represents the sound pressure observed at the outward (FIG. 24) in the D3 direction side (notch part 231b side) of the acoustic signal output apparatus 20. On the other hand, “L25-aaaaa_bbb mm. close SPL c°” represents the sound pressure observed at the outward in the D4 direction side (the side where the notch part 231b is not provided) of the acoustic signal output apparatus 20. A line in which “L25-aaaaa” is “L25-61065” represents the measurement result of the acoustic signal output apparatus 20 provided with the notch part 231b-SW (FIG. 24). A line in which “L25-aaaaa” is “L25-61063” represents the measurement result of the acoustic signal output apparatus 20 provided with the notch part 231b-LW (FIG. 25). A line in which “L25-aaaaa” is “L25-61064” represents the measurement result of the acoustic signal output apparatus 20 provided with the notch part 231b-LN (FIG. 26). “bbb mm” represents the distance from the acoustic signal output apparatus 20 to the measurement position. “c°” represents the direction of the measurement position with respect to the acoustic signal output apparatus 20. When “c°” is 0°, it represents that the direction of the measurement position with respect to the acoustic signal output apparatus 20 is the D1 direction. When “c°” is 90°, it represents that the direction of the measurement position with respect to the acoustic signal output apparatus 20 is orthogonal to the D1-D2 direction. When “c°” is 180°, it represents that the direction of the measurement position with respect to the acoustic signal output apparatus 20 is the D2 direction.

As shown in these, it can be seen that the sound leakage can be adjusted by the size and shape of the notch part 231b.

Note that, in addition to the sound hole 131b, a longitudinally long notch part 231b-LN for opening the inside of the reflector 13 to the outside may be provided at a part of the open end 130 side of the reflector 13.

### [Third Embodiment of Localized Sound Speaker]

A part of the reflector 13 of the localized sound speaker in the first embodiment, modification examples 1 and 2 thereof, and the second embodiment may be used as a diaphragm of a driver unit (second driver unit). By this, the size can be reduced as a whole. Specific example will be shown below.

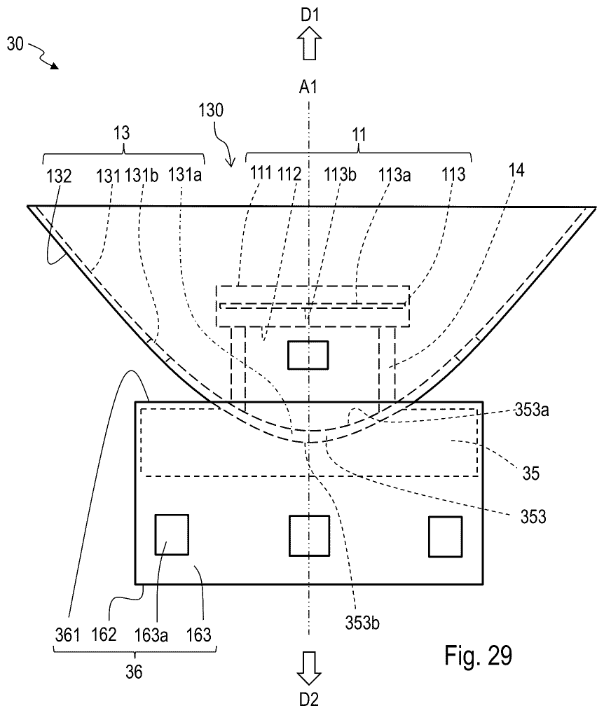

An acoustic signal output apparatus 30 illustrated in FIG. 29 includes a concave type reflector 13 having a paraboloid of revolution or an approximate surface to the paraboloid of revolution inside, driver units 11 and 35 (in other words, speaker driver or driver) for converting an output signal outputted from a reproducing apparatus into an acoustic signal and outputting it, a housing 36 storing the driver unit 35 inside, and a support part 14 for placing the driver unit 11 inside the reflector 13. However, the reflector 13 is placed on a wall part 361 side in the D1 direction of the housing 36, and a bottom part 131a (in a part) of the reflector 13 also functions as a diaphragm 353 of the driver unit 35. That is, the driver unit 35 emits the acoustic signal AC3 (third acoustic signal) from a surface 353a in the D1 direction side (first side) to the D1 direction side (first side) by the vibration of the diaphragm 353 which is a bottom part 131a of the reflector 13, and emits the acoustic signal AC4 (fourth acoustic signal) from the other surface 353b to the D2 direction side (the other side) by the vibration. By this, the size of the acoustic signal output apparatus 30 in the D1-D2 direction can be reduced.

Preferably, at least a part of an inner wall surface 131 of the reflector 13 is the paraboloid of revolution or the surface approximate to the paraboloid of revolution, the paraboloid of revolution has a shape in which a parabola is rotated around the axis line A1 (a specific axis), and the diaphragm 353 is a portion of the bottom part 131a of the reflector 13 placed on the axis line A1 or in the vicinity of the axis line A1. As a result, the sound pressure of the acoustic signal AC3 emitted from the open end 130 of the reflector 13 becomes line-symmetric or approximately line-symmetric with respect to the axis line A1. In addition, it is desirable that the single or a plurality of sound holes 131b (reflector sound holes) is provided at positions excluding the diaphragm 353 of the reflector 13. By this, the acoustic signals AC3, AC4 of high sound pressure can be emitted from the diaphragm 353.

Note that FIG. 29 shows an example in which the driver unit 11 is not stored in the housing 12. However, the driver unit 11 (first driver unit) may be stored in the housing 12 (first housing) different from the housing 36 (second housing), and the housing 12 storing the driver unit 11 inside may be placed inside the reflector 13 (see the modification example 2 of the first embodiment of the localized sound speaker).

### OTHER MODIFICATION EXAMPLES

Note that the disclosed technology is not limited to the embodiments mentioned above. For example, in the first and second embodiments and the modification examples thereof mentioned above, the example in which the bottom part 131a side of the reflector 13 is fixed to the wall part 161 of the housing 16 is shown, but the bottom part 131a side of the reflector 13 may be integrated with the wall part 161 of the housing 16.

In addition, it is desirable that the driver unit 11 is placed at the focal point or in the vicinity of the focal point of the paraboloid of revolution of the reflector 13, but the driver unit 11 may be placed at other positions. For example, the driver unit 11 may be attached to the bottom part 131a side of the reflector 13.

In addition, the reflector 13 may be formed in a horn shape or other shapes.

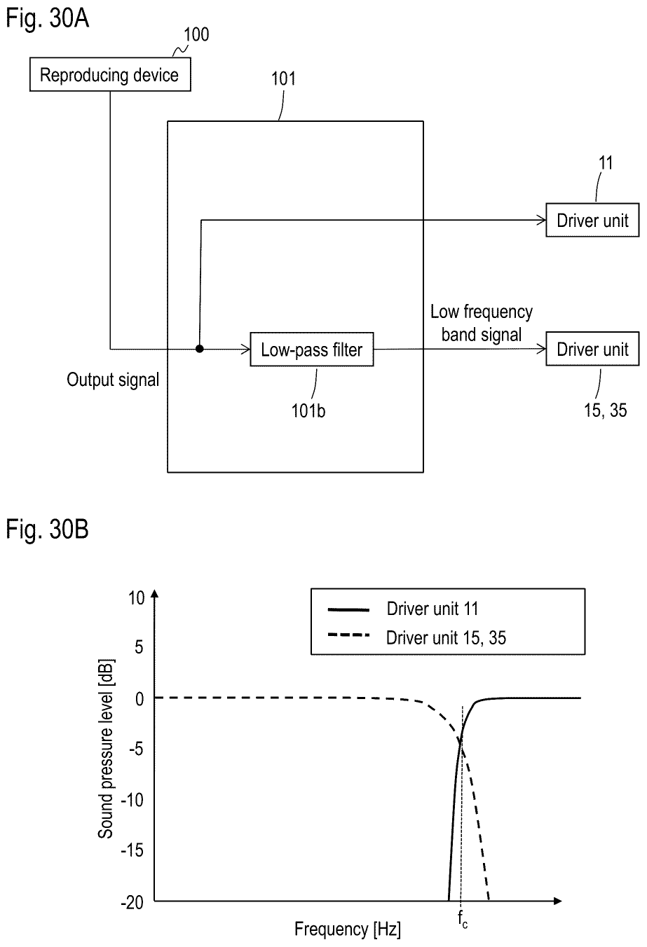

In the above-mentioned embodiments and those modification examples, the high-pass filter 101a may be omitted from the signal separating apparatus 101 illustrated in FIG. 9A. Since the acoustic signals AC1 and AC2 emitted from the driver unit 11 easily cancel each other out by mutual interference in a band on the middle and low band side, a sound pressure level on the middle and low band side by the acoustic signal AC1 and the acoustic signal AC2 at the observation point is lowered. On the other hand, since the acoustic signals AC1 and AC2 do not sufficiently cancel each other out on the high band side, the sound pressure level on the high band side by the acoustic signal AC1 and the acoustic signal AC2 at the observation point is high. This feature plays a role equivalent to the high-pass filter. Therefore, even if the high-pass filter 101a is omitted from the signal separating apparatus 101, the sound pressure level by the acoustic signal AC1 and the acoustic signal AC2 observed at the observation point is suppressed on the middle and low band side and is not suppressed so much on the high band side (FIG. 30B). This effect particularly and remarkably appears when the driver unit 11 is stored in the housing 12 provided with the sound holes 121a and 123a as mentioned above (for example, the modification example 2 of the first embodiment of the localized sound speaker). Therefore, especially when the driver unit 11 is stored in the housing 12 provided with the sound holes 121a and 123a, even if the high-pass filter 101a is omitted, influence on acoustic characteristics is small.

In such a configuration, the output signal outputted from the reproducing apparatus 100 is inputted to the signal separating apparatus 101, and the signal separating apparatus 101 branches the inputted output signal into two signals. The branched output signals are inputted to the driver unit 11 and the low-pass filter 101b, respectively. The driver unit 11 emits the acoustic signal AC1 to the D1 direction side and emits the acoustic signal AC2 to the D2 direction side on the basis of the inputted output signal. The low-pass filter 101b attenuates the high band side of the inputted output signal to obtain and output the low frequency band signal. The low frequency band signal is inputted to either of driver units 15 or 35 of the acoustic signal output apparatus 10 to 30, and the driver unit 15 or 35 emits the acoustic signal AC3 to the D1 direction side and the acoustic signal AC4 to the D2 direction side.

The above description is directed to a speaker suitable as the localized sound speaker of the dangerous area notification apparatus.

1. A dangerous area notification apparatus comprising:
a speaker configured to emit a sound toward a sidewalk or its periphery in a railroad crossing, wherein
the speaker emits a sound different from an alarm sound of a railroad crossing gate.

2. The dangerous area notification apparatus according to claim 1, wherein
the speaker has directivity, and emits a sound strongly transmitted toward a region covering the sidewalk in the railroad crossing or a peripheral region of the sidewalk in the railroad crossing.

3. The dangerous area notification apparatus according to claim 1, wherein
the speaker is a localized sound speaker that can increase sound pressure in a desired region to be higher than sound pressure outside the desired region, and
the desired region is a region covering the sidewalk in the railroad crossing or the peripheral region of the sidewalk in the railroad crossing.

4. The dangerous area notification apparatus according to claim 1, wherein
the sidewalk is provided with tactile paving,
the speaker is a localized sound speaker that can increase sound pressure in the desired region to be higher than sound pressure outside the desired region, and
the desired region is a region covering the tactile paving or the peripheral region of the tactile paving.

5. The dangerous area notification apparatus according to claim 1, comprising:
a control circuitry configured to control contents and/or timing of the sound played from the speaker.

6. The dangerous area notification apparatus according to claim 3, wherein
the localized sound speaker comprises a high frequency speaker driver that generates a sound in a high frequency band, a low frequency speaker driver that generates a sound in a low frequency band, and a reflector having a paraboloid of revolution or a surface approximate to the paraboloid of revolution,
the high frequency speaker driver emits a first acoustic signal of a first phase in an opening direction of the reflector, and emits a second acoustic signal of a second phase in a direction inverse to the opening direction,
the low frequency speaker driver emits a third acoustic signal having the first phase or a phase approximate to the first phase in the opening direction, and emits a fourth acoustic signal having the second phase or a phase approximate to the second phase in a direction inverse to the opening direction, and
the reflector has a first opening for guiding the second acoustic signal to the outside of the reflector and a second opening for guiding the third acoustic signal into the reflector.
